SEQUENCE LISTING (23 sequences)

SEQ ID NO: 1 - DNA, 20 bp, Artificial Sequence
  Synthetic
ccctgttcactgtgagtagg

SEQ ID NO: 2 - DNA, 22 bp, Artificial Sequence
  Synthetic
gcgatccctgaacatgtccatc

SEQ ID NO: 3 - DNA, 22 bp, Artificial Sequence
  Synthetic
agtatctggtaggtgcttccag

SEQ ID NO: 4 - DNA, 20 bp, Artificial Sequence
  Synthetic
caaatagccctggcagattc

SEQ ID NO: 5 - DNA, 20 bp, Artificial Sequence
  Synthetic
tgatacaagggacatcttcc

SEQ ID NO: 6 - DNA, 20 bp, Artificial Sequence
  Synthetic
ttgcctttctctccacaggt

SEQ ID NO: 7 - DNA, 20 bp, Artificial Sequence
  Synthetic
catgtccatcaggttcttgc

SEQ ID NO: 8 - DNA, 23 bp, Artificial Sequence
  Synthetic
tctgtgaagcgagtccttaggct

SEQ ID NO: 9 - DNA, 23 bp, Artificial Sequence
  Synthetic
cgcactctctctctcccaaggaa

SEQ ID NO: 10 - DNA, 20 bp, Artificial Sequence
  Synthetic
aattactgctgaagcccacc

SEQ ID NO: 11 - DNA, 21 bp, Artificial Sequence
  Synthetic
gaatctcctagaactgactgg

SEQ ID NO: 12 - DNA, 27 bp, Artificial Sequence
  Synthetic
aacagttttgagcattttctattgttt

SEQ ID NO: 13 - DNA, 26 bp, Artificial Sequence
  Synthetic
caaagaaaacagaagaaaaataatac

SEQ ID NO: 14 - DNA, 21 bp, Artificial Sequence
  Synthetic
gcgaagagtttgtcctcaacc

SEQ ID NO: 15 - DNA, 21 bp, Artificial Sequence
  Synthetic
aaagtcgctctgagttgttat

SEQ ID NO: 16 - DNA, 18 bp, Artificial Sequence
  Synthetic
cggttccgagacagcaaa

SEQ ID NO: 17 - DNA, 19 bp, Artificial Sequence
  Synthetic
cggttcgatctaggaatgg

SEQ ID NO: 18 - DNA, 21 bp, Artificial Sequence
  Synthetic
ctttgcctcactcaggattgg

SEQ ID NO: 19 - DNA, 21 bp, Artificial Sequence
  Synthetic
actgccacacctccagtcatt

SEQ ID NO: 20 - DNA, 19 bp, Artificial Sequence
  Synthetic
agagtggcctccgctttgt

SEQ ID NO: 21 - DNA, 20 bp, Artificial Sequence
  Synthetic
cgcattggaggacagattca

SEQ ID NO: 22 - DNA, 21 bp, Artificial Sequence
  Synthetic
atgtgtccgtcgtggatctga

SEQ ID NO: 23 - DNA, 22 bp, Artificial Sequence
  Synthetic
atgcctgcttcaccaccttctt

(19) United States
(12) Patent Application Publication
(10) Pub. No.: US 2021/0346328 A1
(43) Pub. Date: Nov. 11, 2021

(54) COMPOSITIONS AND METHODS FOR TREATING METABOLIC DISORDERS

(71) Applicant: The Johns Hopkins University, Baltimore, MD (US)

(72) Inventor: Xu Cao, Baltimore, MD (US)

(21) Appl. No.: 17/278,103

(22) PCT Filed: Sep. 20, 2019
(86) PCT No.: PCT/US2019/052195

§ 371 Date: Mar. 19, 2021

Related U.S. Application Data
(60) Provisional application No. 62/734,604, filed on Sep. 21, 2018.

(51) Int. Cl. A61K 31/192; A61K 31/42; A61K 31/415; A61K 31/5415; A61K 31/616; A61K 31/407; A61K 31/405; A61K 31/12; A61K 31/196; A61K 31/18; A61P 19/02

(52) U.S. Cl. CPC A61K 31/192; A61K 31/42; A61K 31/415; A61K 31/5415; A61K 31/616; A61P 19/02; A61K 31/405; A61K 31/12; A61K 31/196; A61K 31/18; A61K 31/407

(57) ABSTRACT

Provided herein are methods of treating, delaying progression of, or reducing the severity of metabolic disorders characterized with increased COX-2 expression and/or PGE2 expression and/or EP4 expression in bone related cells through administration of an agent configured to inhibit and/or diminish COX-2 expression, and/or PGE2 expression, and/or EP4 expression in bone related cells. In some embodiments, such administration results in one or more of the following: inhibited or reduced COX-2 expression; inhibited or reduced PGE2 expression; inhibited or reduced EP4 expression; inhibited or reduced aberrant subchondral bone remodeling and/or innervation; inhibited or reduced cartilage degeneration; and inhibited or reduced joint destruction.

### FIELD OF THE INVENTION

Provided herein are methods of treating, delaying progression of, or reducing the severity of metabolic disorders characterized with increased COX-2 expression and/or PGE2 expression and/or EP4 expression in bone related cells through administration of an agent configured to inhibit and/or diminish COX-2 expression, and/or PGE2 expression, and/or EP4 expression in bone related cells. In some embodiments, such administration results in one or more of the following: inhibited or reduced COX-2 expression; inhibited or reduced PGE2 expression; inhibited or reduced EP4 expression; inhibited or reduced aberrant subchondral bone remodeling and/or innervation; inhibited or reduced cartilage degeneration; and inhibited or reduced joint destruction.

### INTRODUCTION

Arthritis is a joint disorder that affects 1 or more joints. Osteoarthritis (OA) and rheumatoid arthritis (RA) are the 2 most common forms of arthritis. OA is characterized mainly by joint pain and stiffness, especially affecting the weightbearing joints, such as the knee and hip (see, Busija, L., et al. Clinical rheumatology 24, 757-768 (2010)). Muscle atrophy and joint deformities appear during the advanced stages (see, Buckwalter, J. A. & Martin, J. A. Osteoarthritis. Advanced drug delivery reviews 58, 150-167 (2006)). RA is an autoimmune disease involving chronic synovial inflammation within the joints. Approximately 0.5% to 1% of the population has RA, and small joints are more frequently affected than large joints (see, Smolen, J. S., Aletaha, D. & McInnes, I. B. Rheumatoid arthritis. The Lancet 388, 2023-2038 (2016)). RA is characterized by increased levels of inflammatory cytokines in the synovial joints, including tumor necrosis factor-α (TNF-α), interleukin-6, and interleukin-1 (see, McInnes, I. B. & Schett, G. The New England journal of medicine 365, 2205-2219 (2011)). Currently, arthritis treatment focuses on controlling the symptoms, especially pain; there is no satisfactory prevention or cure. During the end stages of OA and RA, joint replacement is often necessary (see, Smolen, J. S., Aletaha, D. & McInnes, I. B. The Lancet 388, 2023-2038 (2016); Glyn-Jones, S., et al. The Lancet 386, 376-387 (2015)). OA is a costly and prevalent joint disease. OA is predicted to affect 67 million people in the United States by 2030 (see, Sampson, E. R., et al. Science translational medicine 3, 101ra193 (2011)), and in developed countries, its treatment costs between 1.0% and 2.5% of gross domestic product (see, Hiligsmann, M., et al. Seminars in arthritis and rheumatism 43, 303-313 (2013)).

Thus, there is a critical need for improved treatments for arthritis.

The present invention addresses this need.

### SUMMARY OF THE INVENTION

Clinically, many arthritis patients have joint pain and degeneration with no history of trauma or other known cause. Some patients show clear genetic correlations through family history of the disease (see, van Meurs, J. B. Osteoarthritis and cartilage 25, 181-189 (2017)). There has been a major effort to identify potential genetic risk factors for arthritis (see, Styrkarsdottir, U., et al. Nat Genet 49, 801-805 (2017); Evangelou, E., et al. Annals of the rheumatic diseases 73, 2130-2136 (2014); Liu, Y., et al. J Rheumatol 44, 1652-1658 (2017); Valdes, A. M., et al. American journal of human genetics 82, 1231-1240 (2008)). Studying spontaneous OA in animals offers a unique opportunity to identify the factors that may cause joint degeneration. STR/Ort mice are an inbred substrain of STR/N mice (see, Staines, K. A., et al., Osteoarthritis and cartilage 25, 802-808 (2017)). The origin of STR/Ort mice can be traced to 1951, when they were originally established from the NHO strain through 3-methylcholanthrene injection between the F4 and F27 generations (see, Mason, R. M., et al. Osteoarthritis and cartilage 9, 85-91 (2001)). The substrain of STR/Ort mice was developed from the STR/1N strain after initiating inbreeding following a period of non-inbreeding (see, Mason, R. M., et al. Osteoarthritis and cartilage 9, 85-91 (2001)). Notably, the STR/Ort strain is prone to develop OA, which is characterized by subchondral bone sclerosis, osteophyte formation, and articular cartilage degeneration (see, Staines, K. A., et al., Osteoarthritis and cartilage 25, 802-808 (2017). It is worth mentioning that male mice have a higher incidence of OA than female mice (see, Staines, K. A., et al., Osteoarthritis and cartilage 25, 802-808 (2017). Histological lesions of STR/Ort mice closely resemble those of human OA. Previous studies have shown that OA lesions often develop in the medial tibia plateau of the knee joint, rather than the lateral plateau (see, Staines, K. A., et al., Osteoarthritis and cartilage 25, 802-808 (2017). Thus, STR/Ort mice are a promising model of spontaneous OA to study the genetic factors involved in OA.

Sensory nerves are innervated in peripheral tissues, including skin, joint, respiratory, and gastrointestinal tissues, to sense stimuli inside or outside the body, such as pain, temperature, odors, and tastes (see, Pinho-Ribeiro, et al., Trends Immunol 38, 5-19 (2017); Snyder, D. J. & Bartoshuk, L. M. Rev Endocr Metab Disord 17, 149-158 (2016)). The signals collected from sensory nerve endings are processed in the central nervous system to initiate physiological responses. Bone is the largest organ, accounting for more than 80% of body weight. Bone is also an endocrine organ that regulates calcium and mineral metabolism, glucose, fatty acids, and even cancer metastasis by interacting with other tissues (see, Kushwaha, P., et al., Bone, (2017); Engblom, C. et al. Science 358 (2017); Karner, C. M. & Long, F. Bone (2017)). The skeleton has abundant sensory and sympathetic innervations (see, Serre, C. M., et al., Bone 25, 623-629 (1999); Mach, D. B. et al. Neuroscience 113, 155-166 (2002); Hara-Irie, F., et al., Bone 18, 29-39 (1996); Chenu, C. J Musculoskelet Neuronal Interact 4, 132-134 (2004)) and interacts with the central nervous system (see, Oury, F. et al. Genes Dev. 24, 2330-2342, doi:10.1101/gad.1977210 (2010); Ducy, P. et al. Cell 100, 197-207 (2000)). Sympathetic nerves induce catabolic activity in bone through serotonin and cAMP-response element binding protein (CREB) signaling in the hypothalamus (see, Oury, F. et al. Genes Dev. 24, 2330-2342 (2010); Yadav, V. K. et al. Cell 138, 976-989 (2009); Ortuno, M. J. et al. Nat. Med. 22, 1170-1179 (2016)). Specific deletion of sensory nerves in bone impairs bone mass accrual (see, Fukuda, T. et al. Nature 497, 490-493 (2013); Levi, B. Sci Transl Med 9, doi:10.1126/scitranslmed.aan3780 (2017)). Patients with sensory nerve malfunction or loss have an increased bone fracture rate and significantly diminished post-injury bone regeneration (see, Perez-Lopez, L. M., et al., Case Rep Pediatr 2015, 589852; Bar-On, E. et al. J. Bone Joint Surg. Br. 84, 252-257 (2002)). These observations indicate that sensory nerves sense changes in bone density, mechanical stress, and metabolic activity to control bone homeostasis.

Experiments conducted during the course of developing embodiments for the present invention hypothesized the existence of one or more molecules that transmit signals of changes in bone to sensory nerve fibers. Cyclooxygenase activity and prostaglandins are known to mediate skeletal metabolism and inflammation (see, Blackwell, K. A., et al., Trends Endocrinol Metab 21, 294-301 (2010); Bley, K. R., et al., Trends Pharmacol. Sci. 19, 141-147 (1998)). Among prostaglandins, prostaglandin E2 (PGE2) is a multifunctional molecule whose production is controlled by the limiting enzyme cyclooxygenase (COX) (see, Blackwell, K. A., et al., Trends Endocrinol Metab 21, 294-301 (2010)). Evidence shows that PGE2 can elicit primary pain and prolong nociceptor sensitization (see, Kawabata, A. Biol. Pharm. Bull. 34, 1170-1173 (2011); Kuner, R. Nat. Med. 16, 1258-1266, doi:10.1038/nm.2231 (2010)). Non-steroidal anti-inflammatory drugs and COX2 selective inhibitors are the current major medications to treat musculoskeletal pain (see, O'Connor, J. P. & Lysz, T. Drugs Today (Barc) 44, 693-709 (2008)). A multicenter study revealed that COX2 selective inhibitor is associated with lower bone mineral density (BMD) in men; whereas, in postmenopausal women it promotes BMD (see, Salari, P. & Abdollahi, M. Inflamm Allergy Drug Targets 8, 169-175 (2009)), implicating PGE2 in the regulation of bone.

The 15-hydroxyprostaglandin dehydrogenase gene (HPGD) encodes a NAD⁺-dependent 15-hydroxyprostaglandin dehydrogenase (15-PGDH), which catalyzes PGE2. Mutation of this gene impairs the degradation of PGE2 (see, Uppal, S. et al. Nat. Genet. 40, 789-793 (2008)). HPGD mutant mice showed an increased PGE2 level in vivo, which can effectively promote regeneration in different tissues (see, Zhang, Y. et al. Science 348, aaa2340 (2015); Coggins, K. G. et al. Nat. Med. 8, 91-92 (2002); Myung, S. J. et al. Proc. Natl. Acad. Sci. U. S. A. 103, 12098-12102 (2006)). Interestingly, patients with HPGD mutation have presented with subperiosteal new bone formation (see,Yuksel-Konuk, B. et al. Rheumatol. Int. 30, 39-43 (2009)). PGE2 is also potent in stimulating bone formation, and its bone anabolic effect is believed to be through its receptor EP4 in the osteoblasts (see, Blackwell, K. A., et al., Trends Endocrinol Metab 21, 294-301 (2010); Raisz, L. G. & Woodiel, F. N. Prostaglandins Other Lipid Mediat. 71, 287-292 (2003); Minamizaki, T., eta al., Bone 44, 1177-1185 (2009)). However, conditional knockout of the prostaglandin E receptor 4 gene (EP4) in osteoblastic cells did not impair bone density, implying that the bone formation effect of PGE2 does not act through osteoblasts (see, Gao, Q. et al. Bone 45, 98-103 (2009)). In pathological conditions of bone loss during aging or after menopause, the impaired function of sensory nerves and elevated PGE2 level appear simultaneously (see, Burt-Pichat, B. et al. Endocrinology 146, 503-510 (2005); Strotmeyer, E. S. et al. J. Bone Miner. Res. 21, 1803-1810 (2006)). Thus, PGE2-induced pain may reflect its activation of sensory nerves to transmit a “signal” of bone density to maintain bone homeostasis.

Cyclooxygenase-2 (COX-2) inhibitors selectively block the COX-2 enzyme and have been used for treating OA and RA with low risk of adverse gastrointestinal effects (see, Steinmeyer, J. & Konttinen, Y. T. Advanced drug delivery reviews 58, 168-211 (2006)). As noted, blocking this enzyme impedes the production of prostaglandin (PG) E2, which is often the cause of pain and swelling of inflammation (see, Crofford, L. J., et al. Arthritis & Rheumatism 43, 4-13 (2000)). PGs are enzymatically derived metabolites of polyunsaturated fatty acids, such as arachidonic acid. As the most widely produced prostanoid in the human body, PGE2 is a well-known regulator of bone formation (see, Blackwell, K. A., et al., Trends in endocrinology and metabolism: TEM 21, 294-301 (2010)). The crucial limiting step during PGE2 synthesis is the enzyme that catalyzes cyclooxygenase (COX) and peroxidase reactions. There are 2 isoforms of COX: COX-1 is expressed at relatively stable levels in most tissues and is considered “constitutive,” whereas COX-2 is an inducible isoform that is generally expressed at very low levels in most tissues but can be induced to high levels by multiple factors (see, Crofford, L. J., et al. Arthritis & Rheumatism 43, 4-13 (2000)). COX-2 is more efficient at producing PGs than is COX-1 (see, Blackwell, K. A., Raisz, L. G. & Pilbeam, C. C. Trends in endocrinology and metabolism: TEM 21, 294-301 (2010)).

Experiments conducted during the course of development embodiments for the present invention demonstrated that genetically elevated COX-2 expression in the osteocytes of subchondral bone causes both spontaneous OA and rheumatoid arthritis (RA). It was determined that knockout of COX-2 in osteocytes or COX-2 inhibitor treatment effectively rescued the structure of subchondral bone and attenuate cartilage degeneration in spontaneous OA (STR/Ort) and tumor necrosis factor-a transgenic RA. Thus, it was determined that genetically elevated COX-2 expression in subchondral bone induces both OA- and RA-associated joint cartilage degeneration. Inhibition of COX-2 expression could potentially modify joint destruction in patients with arthritis.

Additional experiments investigated whether sensory nerve can sense bone density, metabolic activity, or mechanical stress to control bone homeostasis. It was found that prostaglandin E2 (PGE2) secreted by osteoblastic cells activates PGE2 receptor 4 (EP4) in sensory nerves to regulate bone formation by inhibiting sympathetic activity through the central nervous system. PGE2 secreted by osteoblasts increases when bone density decreases, as demonstrated in osteoporotic and aging animal models. Ablation of sensory nerves erodes the skeletal integrity in two sensory denervation mouse models (TrkA_{Avil}^{-/-}and iDTR_{Avil}^{fl/-}). Specifically, knockout of the EP4 gene in the sensory nerves (EP4_{Avil}^{-/-}) or cyclooxygenase-2 (COX2) (the rate-limiting enzyme for production of PGE2) in the osteoblastic cells (COX2_{OC}^{-/-}) significantly reduced bone volume in adult mice but not young mice during bone development. Sympathetic tone was increased in these two sensory denervation models, and propranolol, a β2-adrenergic antagonist, rescued the bone loss. Furthermore, injection of an inhibitor (SW033291) of PGE2 degradation enzyme 15-hydroxyprostaglandin dehydrogenase (15-PDGH) significantly boosted bone formation, whereas the effect was obstructed in EP4_{Avil}^{-/-} mice. Thus, such experiments demonstrated that PGE2 mediates sensory nerve to control bone homeostasis and promote regeneration.

Additional experiments determined that treatment of OA with COX-2 inhibitors is optimized with different dosage regimens than when used for pain management. For example, it was determined that effective OA treatment was observed with a COX-2 inhibitor dosage regimen of 8 mg/kg for 4 weeks. Quite the contrary, for pain management, COX-2 inhibitor dosage regimen is typically 30 mg/kg for one or two weeks are used. In brief, COX-2 inhibitor celecoxib was gavage fed at different dosages once a day for 4 weeks. Proteoglycan loss and calcification of articular cartilage were effectively attenuated by 8 mg/kg of COX-2 inhibitor in STR/Ort mice relative to controls, as indicated by safranin O and fast green staining; the OARSI score for articular cartilage also improved significantly.

Accordingly, in certain embodiments, the present invention provides methods of treating, delaying progression of, or reducing the severity of metabolic disorders characterized with increased COX-2 expression and/or PGE2 expression and/or EP4 expression in bone related cells, the method comprising administering to a subject in need thereof a therapeutically effective amount of an agent configured to inhibit and/or diminish COX-2 expression, and/or PGE2 expression, and/or EP4 expression in bone related cells. In some embodiments, such administration results in one or more of the following: inhibited or reduced COX-2 expression; inhibited or reduced PGE2 expression; inhibited or reduced EP4 expression; inhibited or reduced aberrant subchondral bone remodeling and/or innervation;

inhibited or reduced cartilage degeneration; and inhibited or reduced joint destruction.

In certain embodiments, the present invention provides methods of inhibiting and/or reducing COX-2 expression in bone cells comprising exposing bone cells characterized with increased COX-2 expression (compared to an established normal expression level) a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing PGE2 expression in bone cells comprising exposing bone cells characterized with increased PGE2 expression (compared to an established normal expression level) a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing EP4 expression in bone cells comprising exposing bone cells characterized with increased EP4 expression (compared to an established normal expression level) a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing aberrant subchondral bone remodeling and/or innervation in bone cells comprising exposing bone cells characterized with aberrant subchondral bone remodeling and/or innervation a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing joint cell destruction in bone cells comprising exposing bone cells characterized with joint destruction a therapeutically effective amount of an agent configured to inhibit and/or diminish COX-2 expression, and/or PGE2 expression, and/or EP4 expression in bone related cells.

In certain embodiments, the agent configured to inhibit and/or diminish COX-2 expression, and/or PGE2 expression, and/or EP4 expression in bone related cells is administered for purposes of reducing COX-2 expression within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing PGE2 expression within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing EP4 expression within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing aberrant subchondral bone remodeling and/or innervation within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing joint destruction within any setting (e.g., in vitro, in vivo, in silico).

Such methods are not limited to particular types or kinds of metabolic disorders characterized with increased COX-2 expression and/or PGE2 expression and/or EP4 expression in bone related cells. In some embodiments, such metabolic disorders include osteoarthritis (OA), rheumatoid arthritis (RA), cardiovascular disease, and diabetes.

Such methods are not limited to particular types or kinds of agents configured to inhibit and/or diminish COX-2 expression, and/or PGE2 expression, and/or EP4 expression in bone related cells.

In some embodiments, the agent configured to inhibit and/or diminish COX-2 expression, and/or PGE2 expression, and/or EP4 expression in bone related cells is a COX-2 inhibiting agent. Non-limiting examples of COX-2 inhibitors that may be used in such methods include celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lornoxicam, nabumetone, and diclofenac, as well as pharmaceutically acceptable salts of each, pharmaceutically acceptable derivatives of each, prodrugs of each, or mixtures thereof.

Such methods are not limited to a specific meaning for a therapeutically effective amount of an agent configured to inhibit and/or diminish COX-2 expression, and/or PGE2 expression, and/or EP4 expression in bone related cells. In some embodiments, a therapeutically effective amount of an agent configured to inhibit and/or diminish COX-2 expression, and/or PGE2 expression, and/or EP4 expression in bone related cells comprises administering approximately 8 mg/kg (e.g., 4 mg/kg, 4.5 mg/kg, 4.6 mg/kg, 5 mg/kg, 5.1 mg/kg, 5.5 mg/kg, 5.76 mg/kg, 5.9 mg/kg, 6.2 mg/kg, 6.5 mg/kg, 6.8 mg/kg, 7.2 mg/kg, 7.4 mg/kg, 7.8 mg/kg, 7.9 mg/kg, 7.98 mg/kg, 8.0001 mg/kg, 8.2 mg/kg, 8.4 mg/kg, 8.42 mg/kg, 8.5 mg/kg, 8.8 mg/kg, 9 mg/kg, 9.2 mg/kg, 9.8 mg/kg, 9.9999 mg/kg, 10.2 mg/kg, 10.31 mg/kg, 10.5 mg/kg, 11 mg/kg, 11.3 mg/kg, 11.8 mg/kg, 12.2 mg/kg, 12.999 mg/kg) for approximately 4 weeks (e.g., 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days).

Such methods are not limited to specific bone related cells. In some embodiments, the bone related cells include osteoblast cells, osteocyte cells, and osteoclast cells. In some embodiments, the bone related cells include any bone cells having aberrant COX-2 expression, and/or PGE2 expression, and/or EP4 expression.

In some embodiments, the subject is a mammalian subject (e.g., mouse, horse, human, cat, dog, gorilla, chimpanzee, etc.). In some embodiments, the subject is a human patient suffering from or at risk of suffering from a metabolic disorder characterized with increased COX-2 expression and/or PGE2 expression and/or EP4 expression in bone related cells.

The present invention contemplates that reducing or inhibiting overexpression of COX-2, PGE-2, and/or EP4 within bone cells satisfies an unmet need for the treatment of metabolic disorders characterized with increased COX-2 expression and/or PGE2 expression and/or EP4 expression in bone related cells. Indeed, use of COX-2 inhibiting agents at dosage regimen of approximately 8 mg/kg for approximately 4 weeks represents a marked change for the use of COX-2 inhibiting agents presently used for pain management.

In some embodiments, the agent is formulated to be administered locally. In some embodiments, the agent configured to inhibit and/or diminish COX-2 expression, and/or PGE2 expression, and/or EP4 expression in bone related cells is formulated to be administered systemically, intravenously, intraarterially, subcutaneously, or intrathecally.

In certain embodiments of the invention, combination treatment with the agent configured to inhibit and/or diminish COX-2 expression, and/or PGE2 expression, and/or EP4 expression in bone related cells and a course of a drug known for treating metabolic disorders characterized with increased COX-2 expression and/or PGE2 expression and/or EP4 expression in bone related cells (e.g., a drug known for treating OA; a drug known for treating RA; a drug known for treating CVS; a drug known for treating diabetes).

The invention also provides pharmaceutical compositions comprising the agent in a pharmaceutically acceptable carrier.

The invention also provides kits comprising one or more agents configured to inhibit and/or diminish COX-2 expression, and/or PGE2 expression, and/or EP4 expression in bone related cells (e.g., agents sufficient to interfere with central nervous system related NK levels and function) and instructions for administering the agent to an animal. The kits may optionally contain one or more other therapeutic agents.

### BRIEF DESCRIPTION OF DRAWINGS

FIG. 1. (A) Determination of targeted-knockout efficiency TrkA gene in TrkA_{Avil}^{-/-} mice by qPCR of mRNA isolated from different type tissue, including dorsal root gangolion (DRG), sympathetic gangolion (Sym G), spinal cord (Spi C), kidney, lung, uterus and spleen. (B) TrkA (Red) and NeuN (Green) double-immunofluorescence images of the femur to confirm the TrkA expression on DRG neurons was ablated in TrkA_{Avil}^{-/-} mice. Scale bar, 20 μm. N=3 per group. **P<0.01 (Student t test).

FIG. 2. Osteoblastic bone formation is reduced without sensory nerve innervation. a Representative images of immunofluorescence staining and quantitative analysis of the CGRP⁺ sensory nerves (green) in the femurs of 12-week-old TrkA^{wt} and TrkA_{Avil}^{-/-} mice. DAPI stains nuclei blue. Scale bar: 100 μm. b Representative micro-computed tomography (μCT) images of femurs from 12-week-old male TrkA^{wt} and TrkA_{Avil}^{-/31} mice. Quantitative analysis of trabecular bone fraction (Tb. BV/TV) and trabecular number (Tb. N). Scale bar: 1 mm. c Histomorphological analysis of osteoblast (N.Ob/B.Pm) and osteoclast (N.Oc/B.Pm) numbers on the trabecular bone surface of femurs of 12-week-old TrkA^{wt} and TrkA_{Avil}^{-/-} mice. d Trichrome staining and quantitative analysis of osteoid surface per bone surface (OS/BS) in femoral bone tissue from 12-week-old TrkA^{wt} and TrkA_{Avil}^{-/-} mice. Scale bar, 50 μm. e ELISA analysis of serum OCN and CTX levels in 12-week-old TrkA^{wt} and TrkA_{Avil}^{-/-} mice. f Representative images of calcein double labeling of trabecular bone of femurs with quantification of mineral apposition rate and bone formation rate in 12-week-old TrkA^{wt} and TrkA_{Avil}^{-/-} mice. Scale bar, 20 μm. g Representative images of immunofluorescence staining and quantitative analysis of the CGRP⁺ sensory nerves (green) in the vertebrae of 12-week-old TrkA^{wt} and TrkA_{Avil}^{-/-} mice. DAPI stains nuclei blue. Scale bar: 100 μm. h Representative μCT images of vertebra from 12-week-old TrkA^{wt} and TrkA_{Avil}^{-/-} mice. Quantitative analysis of trabecular bone fraction (Tb. BV/TV) and trabecular number (Tb. N). Scale bar: 1 mm. i Histomorphological analysis of osteoblast (N.Ob/B.Pm) numbers on the trabecular bone surface of 12-week-old TrkA^{wt} and TrkA_{Avil}^{-/-} mice vertebra. N≥5 per group. *P<0.05, **P<0.01 and N.S. means not significant. (Student t-test)

FIG. 3. Quantative analysis of trabecular bone fraction (Tb. BV/TV) and trabecular number (Tb. N) in 4-week-old TrkA^{wt} and TrkA_{Avil}^{-/-} mice femur. N=5 per group. N.S. means not significant (Student t-test).

FIG. 4. A) Histomorphological analysis of osteoclast (N.Oc/B.Pm) numbers on the trabecular bone surface of vertebrae of 12-week-old TrkA^{wt} and TrkA_{Avil}^{-/-} mice. (B, C) Quantification of mineral apposition rate and bone formation rate in 12-week-old TrkA^{wt} and TrkA_{Avil}^{-/-} mice vertebrae. (D) Histomorphological analysis of osteoclast (N.Oc/B.Pm) numbers on the trabecular bone surface of vertebrae of iDTRAv il+/-mice injected with vehicle or 1 ug per kg per day DTX. (E, F) Quantification of mineral apposition rate and bone formation rate of the vertebral bone in iDTR_{Avil}^{+/-} mice injected with vehicle or DTX. N≥5 per group. *P<0.05, **P<0.01 and N.S. means not significant. (Student test).

Figure. 5. Osteoblastic bone formation is blunted after sensory denervation. a Representative images of immunofluorescence staining and quantitative analysis of the CGRP⁺ sensory nerves in the femurs of 8-week-old iDTR_{Avil}^{+/-} mice injected with vehicle or 1 ug per kg per day DTX 3 time a week for four consecutive weeks. Scale bar: 100 μm. b Representative μCT images of femurs from iDTR_{Avil}^{+/-} mice injected with vehicle or DTX. Quantitative analysis of trabecular bone fraction and trabecular number. Scale bar: 1 mm. c Histomorphological analysis of the osteoblast (N.Ob/B.Pm) and osteoclast (N.Oc/B.Pm) numbers on the trabecular bone surface of femurs of iDTR_{Avil}^{+/-} mice injected with vehicle or DTX. d Representative trichrome staining and quantitative analysis of OS/BS in femoral bone tissue from iDTR_{Avil}^{+/-} mice injected with vehicle or DTX. Scale bar, 50 μm. e ELISA analysis of serum OCN and CTX levels in iDTR_{Avil}^{+/-} mice injected with vehicle or DTX. f Representative images of calcein double labeling of femoral trabecular bone with quantification of MAR and BFR in iDTR_{Avil}^{+/-} mice injected with vehicle or DTX. Scale bar, 20 μm. g Representative images of immunofluorescence staining and quantitative analysis of the CGRP⁺ sensory nerves (green) in the vertebra of iDTR_{Avil}^{+/-} mice injected with vehicle or DTX. DAPI stains nuclei blue. Scale bar: 100 μm. h Representative μCT images of vertebrae from iDTR_{Avil}^{+/-} mice injected with vehicle or DTX. Quantitative analysis of trabecular bone fraction (Tb. BV/TV) and trabecular number (Tb. N). Scale bar: 1 mm. i Histomorphological analysis of osteoblast (N.Ob/B.Pm) numbers on the trabecular bone surface of 12-week-old TrkA^{wt} and TrkA_{Avil}^{-/-} mice vertebra. N≥5 per group. *P<0.05, **P<0.01 and N.S. means not significant. (Student t-test)

FIG. 6. Behavior test of TrkA^{wt} and TrkA_{Avil}^{-/-} and iDTR_{Avil}^{+/-} mice injected with vehicle or DTX. (A, B) The average turn time and the average total time to complete the pole test for TrkA^{wt} and TrkA_{Avil}^{-/-} mice. (C, D) Grip strength of the forelimbs and hindlimbs of TrkA^{wt} and TrkA_{Avil}^{+/-} mice. (E, F) The average turn time and the average total time to complete the pole test for iDTR_{Avil}^{+/-} mice injected with vehicle or DTX. (G, H) Grip strength of the forelimbs and hindlimbs of iDTR_{Avil}^{+/-} mice injected with vehicle or DTX. (N=6 per group. No significant differences per Student tests).

FIG. 7. Elevated PGE2 secretion from osteoblasts with sensory nerve dysfunction. a ELISA analysis of serum PGE2 levels in mice with different treatments, including TrkA^{wt} and TrkA_{Avil}^{-/-} mice; iDTR_{Avil}^{+/-} mice injected with vehicle or DTX; young (2-month-old) and aged (12-month-old) mice; mice with sham or ovariectomy surgery (OVX) for 8 weeks. b Quantitative analysis of the COX2⁺ cells (brown) on trabecular surface of femoral bone from different mice models, including TrkA^{wt} and TrkA_{Avil}^{-/-} mice; iDTR_{Avil}^{+/-} mice injected with vehicle or DTX; young (2-month-old) and aged (12-month-old) mice; and mice with sham or ovariectomy surgery (OVX) for 8 weeks. c Representative images of immunostaining of the COX2⁺ cells (brown) on trabecular surface of femoral bone from mice with sham or OVX for 8 weeks. Scale bar, 20 μm. d, e Representative double-immunofluorescent staining imaged and quantitative analysis of EP4 (red) and CGRP (green) in femurs from mice underwent sham or OVX surgery. Scale bar: 100 μm. f, g Representative double-immunofluorescent staining images and quantitative analysis of EP4 (red) and CGRP (green) in femurs from young and aged mice. Scale bar: 100 μm. N≥5 per group. *P<0.05, **P<0.01 and N.S.

means not significant. (Student t-test)

FIG. 8. (A) Determination of targeted-knockout efficiency of EP4gene in EP4_{Avil}^{-/-} mice by qPCR of mRNA isolated from different types of tissue, including dorsal root gangolion (DRG), sympathetic ganglion (Sym G), spinal cord (Spi C), kidney, lung, uterus, and spleen. (B) EP4 (red) and OCN (green) double-immunofluorescence images of the femur confirm that EP4 expression of osteoblasts was unaffected in EP4_{Avil}^{-/-} mice. Scale bar, 50 μm. N=3 per group. **P<0.01 (Student test).

FIG. 9. Deletion of PGE2 receptor EP4 in sensory nerve results in bone loss. a Double-immunofluorescence images of femoral bone sections from 12-week-old EP4^{wt} or EP4_{Avil}^{-/-} mice using antibodies against EP4 (red) and CGRP (green). DAPI stains nuclei blue. Scale bar, 50 μm. b, c Representative μCT images of femurs from 12-week-old EP4^{wt} and EP4_{Avil}^{+/-} mice. Quantitative analysis of trabecular bone fraction (Tb. BV/TV), trabecular number (Tb. N), cortical thickness (Ct. Th), and cortical bone volume (Cor. BV). d ELISA analysis of serum PGE2 level in 12-week-old EP4^{wt} and EP4_{Avil}^{-/-} mice. e Representative images of immunostaining and quantitative analysis of the COX2⁺ cells (in brown) on trabecular bone surface of femoral bone from 12-week-old EP4^{wt} and EP4_{Avil}^{-/-} mice. Scale bar, 20 μm. f Histomorphological analysis of osteoblast (N.Ob/B.Pm) and osteoclast (N.Oc/B.Pm) numbers on the trabecular bone surface of femurs of 12-week-old EP4^{wt} and EP4_{Avil}^{-/-} mice. g ELISA analysis of serum OCN and CTX levels in 12-week-old EP4^{wt} and EP4_{Avil}^{-/-} mice. h Representative trichrome staining and quantitative analysis of osteoid surface per bone surface (OS/BS) in femoral bone tissue of 12-week-old EP4^{wt} and EP4_{Avil}^{-/-} mice. Scale bar, 50 μm. i, j Ten-week-old EP4^{wt} and EP4_{Avil}^{-/-} mice were injected with vehicle or 3 mg per kg per day PGE2 for 3 consecutive days, and bone samples were harvested 12 days after injection. Calcein was injected 5 days and 1 day before sacrifice. Representative images of calcein double labeling of femoral trabecular bone with quantification of mineral apposition rate (MAR) and bone formation rate (BFR). Scale bar, 20 μm. N≥5 per group. *P<0.05, **P<0.01 and N.S. means not significant. (Student t-test, except j with ANOVA)

FIG. 10. Quantative analysis of trabecular bone fraction (Tb. BV/TV) and trabecular number (Tb. N) in 4-week-old EP4_{wt} and EP4_{Avil}^{-/-} mice femur. N=5 per group. N.S. means not significant (Student t test)

FIG. 11. The average turn time (A) and average total time (B) to complete the pole test for EP4_{wt} and EP4_{Avil-/-}-mice. Grip strength of the forelimbs (C) and hindlimbs (D) of EP4_{wt} and EP4_{Avil-/-}-mice. (N≥7 per group. No significant differences per Student t tests).

FIG. 12. (A) EP4 (red) and OCN (green) double-immunofluorescence images of the femur to confirm that the EP4 was no longer expressed in the osteoblasts of EP4_{OC-/-} mice. Scale bar, 50 μm. (B) Quantitative analysis of trabecular bone fraction (Tb. BV/TV) and trabecular number (Tb. N) of the femurs from EP4^{wt} and EP4_{OC-/-} mice. (N=5 per group. No significant differences per Student t tests).

FIG. 13. Behavior test of COX2^{wt} and COX2_{OC}^{-/-} mice. (A, B) The average turn time and the average total time to complete the pole test for COX2^{wt} and COX2_{OC}^{-/-} mice. (C, D) Grip strength of the forelimbs and hindlimbs of COX2^{wt} and COX2_{OC}^{-/-} mice. (N≥6 per group. No significant differences on Student t tests).

FIG. 14. Ablation of COX2 in osteoblasts leads to reduced bone formation. a Representative μCT images of the femurs of 12-week-old COX2^{wt} and COX2_{OC}^{-/-} mice. Quantitative analysis of the trabecular bone fraction (Tb. BV/TV) and trabecular number (Tb. N). Scale bar: 1 mm. b Quantitative analysis of cortical thickness (Ct. Th) and cortical bone volume (Cor. BV). c Representative images of immunostaining and quantitative analysis of the COX2⁺ cells (brown) on trabecular bone surface of femoral bone from 12-week-old COX2^{wt} and COX2_{OC}^{-/-} mice. Scale bar, 20 μm. d ELISA analysis of the serum and bone marrow PGE2 in 12-week-old COX2^{wt} and COX2_{OC}^{-/-} mice. e Histomorphological analysis of osteoblast (N.Ob/B.Pm) and osteoclast (N.Oc/B.Pm) numbers on the trabecular bone surface of 12-week-old COX2^{wt} and COX2_{OC}^{-/-} mice. f Quantitative analysis of the trabecular bone fraction (Tb. BV/TV) and trabecular number (Tb. N) of the femurs from COX2^{wt} and COX_{Dmp-1}^{-/-} mice. N≥5 per group. *P<0.05, **P<0.01 and N.S. means not significant. (Student t-test).

FIG. 15. Quantative analysis of trabecular bone fraction (Tb. BV/TV) and trabecular number (Tb. N) in 4-week-old COX2^{wt} andCOX2OC^{-/-} mice femur. N=5 per group. N.S. means not significant (Student t test).

FIG. 16. Aberrant subchondral bone formation in spontaneous OA mice. (a) Top, safranin O and fast green staining of sagittal sections of the tibia medial subchondral bone of STR/Ort (STR) and CBA control mice at 2, 4, and 6 months old, proteoglycan (red) and bone (blue). Scale bar, 200 μm. Bottom; hematoxylin and eosin staining of the subchondral bone plate and cartilage. The hyaline cartilage (HC) and calcified cartilage (CC) thicknesses are indicated by double-headed arrows. Scale bars, 100 μm. (b) Immunohistochemical staining for MMP13 (brown) in the articular cartilage of STR/ORT and CBA mice at 4 months old. Scale bars, 100 μm. (c) Quantitative analysis of the proportion of MMP13⁺ cells in the articular cartilage of STR/ORT and CBA mice at 4 months old. N=5 per group. (d) Osteoarthritis Research Society International scores of STR/ORT and CBA control mice at 2, 4, and 6 months old. N=5 per group. (e) Representative μCT images of transverse, coronal, and sagittal views of the tibia subchondral bone of 4-month-old STR/ORT and CBA mice. (f, g) Quantitative analysis of bone volume (BV) per tissue volume (TV) (f) and trabecular pattern factor (Tb.Pf) (g) in subchondral bone determined by μCT analysis. N=6 per group. All data shown as mean±standard deviation. *P<0.05 compared with the CBA control group at the corresponding time points. Statistical significance was determined by Student t-test.

FIG. 17. Uncoupled subchondral bone remodeling is not involved in spontaneous osteoarthritis. (a) Images of TRAP staining and immunohistochemical staining of pSmad2/3⁺ cells, osteocalcin⁺ cells and osterix⁺ cells (brown) in subchondral bone from STR/ORT mice and CBA controls at 2, 4, and 6 months old. Scale bars, 50 μm. (b) Quantitative analysis of TRAP⁺ cells in bone surface of subchondral bone from STR/ORT mice and CBA controls. N=5 per group. (c) Quantitative analysis of pSmad2/3⁺ cells in the subchondral bone of STR/ORT mice and CBA controls at 2, 4, and 6 months old. N=5 per group. (d) Quantitative analysis of osteocalcin⁺ cells in the subchondral bone of STR/ORT and CBA mice. N=5 per group. (e) Quantitative analysis of osterix⁺ cells in the subchondral bone of STR/ORT and CBA mice. N=5 per group. (f-h) Representative images of calcein double-labeling of subchondral bone (f) with quantification of mineral apposition rate (MAR) (g) and bone formation rate (BFR) per bone surface (BS) (h) in STR/ORT mice and CBA controls at 6 months old. Scale bar, 25 μm. N=3 per group. All data shown as mean±standard deviation. *P<0.05, compared with the CBA control group at the corresponding time points. Statistical significance was determined by Student t-test.

FIG. 18. Elevated COX-2 activity in the subchondral bone of spontaneous OA. (a) Immunohistochemical staining for COX-2 (brown) in the subchondral bone of STR/ORT and CBA mice at 2, 4, and 6 months old. Scale bars, 50 μm. (b) Quantitative analysis of COX-2⁺ cells (per mm²) in subchondral bone of STR/ORT and CBA mice. N=5 mice in each group from three independent experiments. (c) Western blot analysis of COX-2 protein expression in osteocytes isolated from subchondral bone of STR/ORT and CBA mice at 4 months old. (d) PGE2 levels in medium used to culture the osteocytes isolated from subchondral bone of STR/ORT and CBA mice at 4 months old. N=5 mice in each group from three independent experiments. (e) Serum PGE2 levels of 4-month-old STR/ORT and CBA mice. N=5 mice in each group from three independent experiments. (f-g) Immunohistochemical staining (0 and quantitative analysis (g) of COX-2⁺ cells (brown) in the subchondral bone of C57BL/6 mice 30 days after sham operation or anterior cruciate ligament transection (ACLT) surgery. Scale bar, 50 μm. N=5 mice in each group from three independent experiments. (h-i) Immunohistochemical staining (h) and quantitative analysis (i) of COX-2⁺ cells (brown) in the articular cartilage of STR/ORT and CBA mice at 4 months old. Scale bars, 50 μm. N=5 mice in each group from three independent experiments. (j-k) Immunohistochemical staining (j) and quantitative analysis (k) of COX-2⁺ cells (brown) in the synovial membrane of STR/ORT and CBA mice at 4 months old. Scale bars, 50 μm. N=5 mice in each group from three independent experiments. (1) Quantitative analysis of mechanical allodynia of 4-month-old STR/ORT and CBA mice measured by foot-lift response frequency to stimulation with 0.008-g von Frey filament. N=5 mice in each group from three independent experiments. All data shown as mean±standard deviation. *P<0.05. Statistical significance was determined by Student t-test.

FIG. 19. Elevated COX-2 expression in cortical bone and trabecular bone of STR/ort mice. (a-b) Immunohistochemical staining (a) and quantitative analysis (b) of COX-2⁺ cells (brown) in tibia cortical bone of 6 months old STR/ort and CBA mice. Scale bars, 50 N=5 per group. (c-d) Immunohistochemical staining (c) and quantitative analysis (d) of COX-2⁺ cells (brown) in tibia trabecular bone of 6 months old STR/ort and CBA mice. Scale bars, 50 μm. N=5 per group. All data shown as mean±standard deviation. *P<0.05; **P<0.01. Statistical significance was determined by Student t-test.

FIG. 20. Elevated COX-2 Activity in the Subchondral Bone of Genetically Modified RA and both human OA and RA. (a-b) Immunohistochemical staining (a) and quantitative analysis (b) of COX-2⁺ cells (brown) in subchondral bone of type II collagen-induced arthritis mice models (CIA) and their nonimmunized controls (NIC); TNF-α transgenic mice (TNF-α Tg^{+/-}) and their wild type (WT) controls at 4 months old. Scale bars, 100 μm. N=5 per group. (c) Western blot analysis of COX-2 protein expression in isolated osteocytes from subchondral bone of TNF-α Tg^{+/-} mice and their WT controls at 4 months old. (d) Serum PGE2 levels of 4-month-old TNF-α Tg^{+/-} mice and their WT controls. (e) Immunohistochemical staining for COX-2 in subchondral bone of OA patients, RA patients, and their healthy controls. Scale bars, 100 μm. (f) The proportion of subchondral bone sample with high COX-2 expression (more than 20% osteocytes were considered COX-2⁺). (g) The percentage of COX-2⁺ osteocytes in subchondral bone samples with high COX-2 expression from 13 OA patients and 6 healthy controls. All data shown as mean±standard deviation. *P<0.05. Statistical significance was determined by Student t-test.

FIG. 21. Uncoupled subchondral bone remodeling was increased in TNF-α transgenic RA mice. (a) Representative images of TRAP staining, immunohistochemical staining for osteocalcin (OCN) and osterix (OSX) in subchondral bone marrow of TNF-α Tg^{+/-} mice and their wild type (WT) controls at 4 months old. Scale bars, 25 μm. (b) Quantitative analysis of TRAP⁺ cells in bone surface of TNF-α Tg^{+/-} mice and their WT controls at 4 months old. (c) Quantitative analysis of OCN⁺ cells in bone surface of TNF-α Tg^{+/-} mice and their WT controls at 4 months old. (d) Quantitative analysis of OSX⁺ cells (per mm²) in subchondral bone marrow of TNF-α Tg^{+/-} mice and their WT controls at 4 months old. All data shown as mean±standard deviation. *P<0.05. Statistical significance was determined by Student t-test.

FIG. 22. Inhibition of COX-2 attenuates progression of RA. (a) Safranin O and fast green staining of sagittal sections of the tibia medial subchondral bone of COX-2^{flox/flox}, TNF-α Tg^{+/-}-COX-2^{flox/flox}, and TNF-α Tg^{+/-}DMP1-Cre:: COX-2^{flox/flox} mice. Scale bars, 200 μm. (b-c) Immunohistochemical staining (b) and quantitative analysis (c) of COX-2⁺ cells (brown) in subchondral bone of COX-2^{flox/flox}, TNF-α Tg^{+/-}-COX-2^{flox/flox}, and TNF-α Tg^{+/-} DMP1-Cre:: COX-2^{flox/flox} mice. Scale bars, 25 μm. N=5 mice in each group from three independent experiments. (d-e) Quantitative analysis of bone volume (BV) per tissue volume (TV) (d) and trabecular pattern factor (Tb.Pf) (e) in subchondral bone of COX-2^{flox/flox}, TNF-α Tg^{+/-}-COX-2^{flox/flox}, and TNF-α Tg^{+/-} DMP1-Cre:: COX-2^{flox/flox} mice determined by μCT analysis. N=5 mice in each group from three independent experiments. All data shown as mean±standard deviation. *P<0.05; **P<0.01. Statistical significance was determined by ANOVA.

FIG. 23. Conditional knockout of COX-2 in osteocytes of TNF-α transgenic RA mice rescues uncoupled subchondral remodeling. (a) Representative images of TRAP staining, immunohistochemical staining for osteocalcin (OCN) and osterix (OSX) in subchondral bone of COX-2^{flox/flox}, TNF-α Tg^{+/-}-COX-2^{flox/flox}, and TNF-α Tg^{+/-} DMP1-Cre:: COX-2^{flox/flox} mice. Scale bars, 25 μm. (b-d) Quantitative analysis of TRAP⁺ cells (b), OCN⁺ cells (c), and OSX⁺ cells (d) in subchondral bone of COX-2^{flox/flox}, TNF-α Tg^{+/-}-COX-2^{flox/flox}, and TNF-α Tg^{+/-} DMP1-Cre:: COX-2^{flox/flox} mice. N=5 per group. All data shown as mean±standard deviation. *P<0.05; **P<0.01. Statistical significance was determined by ANOVA.

FIG. 24. Inhibition of COX-2 in TNF-α transgenic RA mice rescues uncoupled subchondral remodeling. (a) Safranin O and fast green staining of sagittal sections of the tibia medial subchondral bone of Tg^{+/-} mice treated with vehicle (Vehicle) and TNF-α Tg^{+/-} mice treated with COX-2 inhibitor (Inhibitor). Scale bars, 200 μm. (b) Serum PGE2 levels of TNF-α transgenic RA mice treated with vehicle or COX-2 inhibitor. N=5 per group. (c-d) Quantitative analysis of bone volume (BV) per tissue volume (TV) (c) and trabecular pattern factor (Tb.Pf) (d) in subchondral bone of Vehicle and Inhibitor groups. N=5 per group. (e) Representative images of TRAP staining and immunohistochemical staining for OCN and OSX in subchondral bone of Vehicle, Inhibitor and wild type (WT) controls. Scale bars, 25 μm. (f-h) Quantitative analysis of TRAP⁺ cells (f), OCN⁺ cells (g), and OSX⁺ cells (h) in subchondral bone of Vehicle, Inhibitor and wild type (WT) controls. N=5 per group. All data shown as mean±standard deviation. *P<0.05; **P<0.01. Statistical significance was determined by Student t-test for (b-d). Statistical significance was determined by ANOVA for (f-h).

FIG. 25. Inhibition of COX-2 attenuates spontaneous OA progression in STR/ORT mice. (a) Top, safranin O and fast green staining of subchondral bone of STR/ORT mice and their CBA control mice treated with vehicle or COX-2 inhibitor, proteoglycan (red) and bone (blue). Scale bar, 200 μm. Medium, representative images of TRAP staining in subchondral bone of STR/ORT and CBA mice treated with vehicle or COX-2 inhibitor. Scale bars, 50 μm. Bottom, trichrome staining of subchondral bone of STR/ORT and CBA mice treated with vehicle or COX-2 inhibitor. Scale bars, 50 μm. (b) OARSI scores of STR/ORT mice and their CBA control mice treated with vehicle or COX-2 inhibitor. N=5 per group. (c) Quantitative analysis of TRAP⁺ cells in bone surface of STR/ORT mice and their CBA control mice treated with vehicle or COX-2 inhibitor. N=5 per group. (d) Serum PGE2 levels of STR/ORT mice and their CBA control mice treated with vehicle or COX-2 inhibitor. N=5 per group. (e-g) Representative images of calcein double-labeling of subchondral bone (e) with quantification of mineral apposition rate (MAR) (f) and bone formation rate (BFR) per bone surface (BS) (g) in STR/ORT mice treated with vehicle or COX-2 inhibitor. Scale bar, 25 μm. (h-i) Quantitative analysis of bone volume (BV) per tissue volume (TV) (h) and trabecular pattern factor (Tb.Pf) (i) in subchondral bone determined by μCT analysis. N=5 per group. (j) Quantitative analysis of mechanical allodynia of STR/ORT mice treated with vehicle or COX-2 inhibitor measured by foot-lift response frequency to stimulation with 0.008-g von Frey filament. N=5 per group. All data shown as mean±standard deviation. *P<0.05. Statistical significance was determined by Student t-test.

FIG. 26. Double-immunofluorescence images of femoral bone sections of 12-week-old C57BL/6 mice using antibodies against CGRP (green) and OCN (red). DAPI stains nuclei blue. Scale bar, 20 μm

FIG. 27. PGE2 stimulates hypothalamic CREB signaling through sensory nerve. a Representative images of DRG neurons isolated from EP4^{wt} and EP4_{Avil}^{-/-} mice pre-incubated with vehicle or 10 μM PGE2 for 5 min, and subsequently treated with calcium imaging buffer (with calcium loaded). The red dots represent activated DRG neurons, and the green dots represent resting DRG neurons. Scale bar, 50 μm. Quantitative analysis was performed with results from three independent assays. b Double-immunofluorescence images of hypothalamus tissue sections from 12-week-old EP4^{wt} or EP4_{Avil}^{-/-} mice with vehicle or 3 mg per kg PGE2 treatment for 6 h using antibodies against CREB (red) and p-CREB (green). DAPI stains nuclei blue. Scale bar, 20 μm. c qRT-PCR analysis of UCP1 expression in adipose tissue and ELISA evaluation of epinephrine level of the serum from EP4^{wt} and EP4_{Avil}^{-/-} mice. d qRT-PCR analysis of UCP1 expression in adipose tissue and ELISA evaluation of epinephrine level of the serum from COX2^{wt} and COX2_{OC}^{-/-} mice. e, f Representative images of immunostaining of the femoral bone tissue sections from EP4^{wt} and EP4_{Avil}^{-/-} and COX2^{wt} and COX2_{OC}^{-/-} mice with antibody against OCN. Scale bar, 50 μm. Projection length of the OCN⁺ lining cells was measured. g, h Double-immunofluorescence images of femoral bone tissue sections from 12-week-old COX2^{wt} and COX2_{OC}^{-/-} and EP4^{wt} and EP4_{Avil}^{-/-} mice using antibodies against OSX (red) and Ki67 (green). DAPI stains nuclei blue. Scale bar, 20 μm. Percentage of Osx- and Ki67-double positive cells and the number of OSX positive cells per trabecular bone surface were quantified. i 8-week-old male EP4^{wt} and EP4_{Avil}^{-/-} mice were injected with low dose (0.5 mg per kg per day) propranolol for 6 weeks. Representative images and quantitative analysis of the μCT images of femurs. Scale bar: 1 mm. N≥5 per group. *P<0.05, **P<0.01 and N.S. means not significant. (Student t-test for b-h, ANOVA for a-i)

FIG. 28. (A) Western blot analysis of HTR2C and p-CREB in hypothalamus tissue from 12-week-old mice after PGE2 or vehicle injection at different periods. (B) Double-immunofluorescence images of hypothalamus tissue sections from C57BL/6mice with vehicle, EP1/3 agonist (200 ug per kg) or EP4 agonist (200 ug per kg) treatment for 6 hours using antibodies against CREB (red) and p-CREB (green). DAPI stains nuclei blue. Scale bar, 50 μm.

FIG. 29. 8-week-old male COX2^{wt} andCOX2_{OC}^{-/-} mice were injected with low does (0.5 mg per kg per day) propranolol for 6 weeks. Representative images and quantitative analysis of the μCT images of femurs. N=5 per group. *P<0.05, **P<0.01 and N.S. means not significant. (ANOVA).

FIG. 30. (A) ELISA evaluation of bone marrow PGE2 concentrations (pg per ml) in control and loading applied mice. (B) Western blot analysis of COX2 expression change in osteoblastic MC3T3 cells treated with different loading force. (C) ELISA evaluation of bone marrow PGE2 concentrations (pg per ml) in control and unloading treated COX2_{OC}^{-/-}-mice. N=4 per group. *P<0.05, **P<0.01 and N.S. means not significant. (Studentttest and ANOVA).

FIG. 31. PGE2 promotes regeneration through sensory nerve. a μCT analysis of bone regeneration after femoral bone marrow ablation in 12-week-old EP4^{wt} and EP4_{Avil}^{-/-} mice treated with 10 mg per kg per day SW033291 or vehicle. Scale bar: 1 mm. Selected areas for the measurements of bone volume (BV)/tissue volume (TV) were indicated with a yellow square. b Representative images of hematoxylin-eosin staining, double immunofluorescence analysis of CD31⁺ Emcn⁺ cells, and immunofluorescence analysis of Leptin receptor (LepR)⁺ cells in the regeneration area. Scale bar, 100 μm. c Graphic illustration of this study. When bone density decreases by osteoclast bone resorption, PGE2 secretion by osteoblastic cells increases at bone remodeling sites. PGE2 activates EP4 receptor at sensory nerve to tune down sympathetic tones for osteoblast differentiation at the bone remodeling microenvironment. The sensory nerve controlling process is likely a temporal-spatial precision action. N≥5 per group. *P<0.05, **P<0.01 and N.S. means not significant. (ANOVA)

FIG. 32. (A) Double-immunofluorescence images of liver tissue sections from TrkA^{wt} and TrkA_{Avil}^{-/-} mice with vehicle or SW033291 treatment using antibodies against and Ki67 (red) and CGRP (green). DAPI stains nuclei blue. Percentages of the Ki67+cells are shown in the right panel. Scale bar, 100 μm.(B) BrdU staining images of liver tissue sections from TrkA^{wt} and TrkA_{Avil}^{-/-} mice with vehicle or SW033291 treatment. Scale bar, 100 mm. Percentages of the BrdU+cells are shown in the right panel. N=4 per group. *P<0.05, **P<0.01 and N.S. means not significant. (ANOVA).

### DETAILED DESCRIPTION OF THE INVENTION

Experiments conducted during the course of development embodiments for the present invention demonstrated that genetically elevated COX-2 expression in the osteocytes of subchondral bone causes both spontaneous OA and rheumatoid arthritis (RA). It was determined that knockout of COX-2 in osteocytes or COX-2 inhibitor treatment effectively rescued the structure of subchondral bone and attenuate cartilage degeneration in spontaneous OA (STR/Ort) and tumor necrosis factor-a transgenic RA. Thus, it was determined that genetically elevated COX-2 expression in subchondral bone induces both OA- and RA-associated joint cartilage degeneration. Inhibition of COX-2 expression could potentially modify joint destruction in patients with arthritis.

Additional experiments investigated whether sensory nerve can sense bone density, metabolic activity, or mechanical stress to control bone homeostasis. It was found that prostaglandin E2 (PGE2) secreted by osteoblastic cells activates PGE2 receptor 4 (EP4) in sensory nerves to regulate bone formation by inhibiting sympathetic activity through the central nervous system. PGE2 secreted by osteoblasts increases when bone density decreases, as demonstrated in osteoporotic and aging animal models. Ablation of sensory nerves erodes the skeletal integrity in two sensory denervation mouse models (TrkA_{Avil}^{-/-} and iDTR_{Avil}^{fl/-}). Specifically, knockout of the EP4 gene in the sensory nerves (EP4_{Avil}^{-/-}) or cyclooxygenase-2 (COX2) (the rate-limiting enzyme for production of PGE2) in the osteoblastic cells (COX2_{OC}^{-/-}) significantly reduced bone volume in adult mice but not young mice during bone development. Sympathetic tone was increased in these two sensory denervation models, and propranolol, a β2-adrenergic antagonist, rescued the bone loss. Furthermore, injection of an inhibitor (SW033291) of PGE2 degradation enzyme 15-hydroxyprostaglandin dehydrogenase (15-PDGH) significantly boosted bone formation, whereas the effect was obstructed in EP4_{Avil}^{-/-} mice. Thus, such experiments demonstrated that PGE2 mediates sensory nerve to control bone homeostasis and promote regeneration.

Additional experiments determined that treatment of OA with COX-2 inhibitors is optimized with different dosage regimens than when used for pain management. For example, it was determined that effective OA treatment was observed with a COX-2 inhibitor dosage regimen of 8 mg/kg for 4 weeks. Quite the contrary, for pain management, COX-2 inhibitor dosage regimen is typically 30 mg/kg for one or two weeks are used. In brief, COX-2 inhibitor celecoxib was gavage fed at different dosages once a day for 4 weeks. Proteoglycan loss and calcification of articular cartilage were effectively attenuated by 8 mg/kg of COX-2 inhibitor in STR/Ort mice relative to controls, as indicated by safranin O and fast green staining; the OARSI score for articular cartilage also improved significantly.

Accordingly, provided herein are methods of treating, delaying progression of, or reducing the severity of metabolic disorders characterized with increased COX-2 expression and/or PGE2 expression and/or EP4 expression in bone related cells through administration of an agent configured to inhibit and/or diminish COX-2 expression, and/or PGE2 expression, and/or EP4 expression in bone related cells. In some embodiments, such administration results in one or more of the following: inhibited or reduced COX-2 expression; inhibited or reduced PGE2 expression; inhibited or reduced EP4 expression; inhibited or reduced aberrant subchondral bone remodeling and/or innervation; inhibited or reduced cartilage degeneration; and inhibited or reduced joint destruction.

Accordingly, in certain embodiments, the present invention provides methods of treating, delaying progression of, or reducing the severity of metabolic disorders characterized with increased COX-2 expression and/or PGE2 expression and/or EP4 expression in bone related cells, the method comprising administering to a subject in need thereof a therapeutically effective amount of an agent configured to inhibit and/or diminish COX-2 expression, and/or PGE2 expression, and/or EP4 expression in bone related cells. In some embodiments, such administration results in one or more of the following: inhibited or reduced COX-2 expression; inhibited or reduced PGE2 expression; inhibited or reduced EP4 expression; inhibited or reduced aberrant subchondral bone remodeling and/or innervation; inhibited or reduced cartilage degeneration; and inhibited or reduced joint destruction.

In certain embodiments, the present invention provides methods of inhibiting and/or reducing COX-2 expression in bone cells comprising exposing bone cells characterized with increased COX-2 expression (compared to an established normal expression level) a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing PGE2 expression in bone cells comprising exposing bone cells characterized with increased PGE2 expression (compared to an established normal expression level) a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing EP4 expression in bone cells comprising exposing bone cells characterized with increased EP4 expression (compared to an established normal expression level) a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing aberrant subchondral bone remodeling and/or innervation in bone cells comprising exposing bone cells characterized with aberrant subchondral bone remodeling and/or innervation a therapeutically effective amount of an agent configured to inhibit and/or diminish COX-2 expression, and/or PGE2 expression, and/or EP4 expression in bone related cells.

In certain embodiments, the present invention provides methods of inhibiting and/or reducing joint cell destruction in bone cells comprising exposing bone cells characterized with joint destruction a therapeutically effective amount of an agent configured to inhibit and/or diminish COX-2 expression, and/or PGE2 expression, and/or EP4 expression in bone related cells.

In certain embodiments, the agent configured to inhibit and/or diminish COX-2 expression, and/or PGE2 expression, and/or EP4 expression in bone related cells is administered for purposes of reducing COX-2 expression within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing PGE2 expression within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing EP4 expression within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing aberrant subchondral bone remodeling and/or innervation within any setting (e.g., in vitro, in vivo, in silico).

In certain embodiments, the agent configured to inhibit and/or diminish COX-2 expression, and/or PGE2 expression, and/or EP4 expression in bone related cells is administered for purposes of reducing joint destruction within any setting (e.g., in vitro, in vivo, in silico).

Such methods are not limited to particular types or kinds of metabolic disorders characterized with increased COX-2 expression and/or PGE2 expression and/or EP4 expression in bone related cells. In some embodiments, such metabolic disorders include osteoarthritis (OA), rheumatoid arthritis (RA), cardiovascular disease, and diabetes.

Such methods are not limited to particular types or kinds of agents configured to inhibit and/or diminish COX-2 expression, and/or PGE2 expression, and/or EP4 expression in bone related cells.

In some embodiments, the agent configured to inhibit and/or diminish COX-2 expression, and/or PGE2 expression, and/or EP4 expression in bone related cells is a COX-2 inhibiting agent. Non-limiting examples of COX-2 inhibitors that may be used in such methods include celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lornoxicam, nabumetone, and diclofenac, as well as pharmaceutically acceptable salts of each, pharmaceutically acceptable derivatives of each, prodrugs of each, or mixtures thereof.

Such methods are not limited to a specific meaning for a therapeutically effective amount of an agent configured to inhibit and/or diminish COX-2 expression, and/or PGE2 expression, and/or EP4 expression in bone related cells. In some embodiments, a therapeutically effective amount of an agent configured to inhibit and/or diminish COX-2 expression, and/or PGE2 expression, and/or EP4 expression in bone related cells comprises administering approximately 8 mg/kg for approximately 4 weeks.

Such methods are not limited to specific bone related cells. In some embodiments, the bone related cells include osteoblast cells, osteocyte cells, and osteoclast cells. In some embodiments, the bone related cells include any bone cells having aberrant COX-2 expression, and/or PGE2 expression, and/or EP4 expression.

In some embodiments, the subject is a mammalian subject (e.g., mouse, horse, human, cat, dog, gorilla, chimpanzee, etc.). In some embodiments, the subject is a human patient suffering from or at risk of suffering from a metabolic disorder characterized with increased COX-2 expression and/or PGE2 expression and/or EP4 expression in bone related cells.

The present invention contemplates that reducing or inhibiting overexpression of COX-2, PGE-2, and/or EP4 within bone cells satisfies an unmet need for the treatment of metabolic disorders characterized with increased COX-2 expression and/or PGE2 expression and/or EP4 expression in bone related cells. Indeed, use of COX-2 inhibiting agents at dosage regimen of approximately 8 mg/kg for approximately 4 weeks represents a marked change for the use of COX-2 inhibiting agents presently used for pain management.

In some embodiments, the agent is formulated to be administered locally. In some embodiments, the agent configured to inhibit and/or diminish COX-2 expression, and/or PGE2 expression, and/or EP4 expression in bone related cells is formulated to be administered systemically, intravenously, intraarterially, subcutaneously, or intrathecally.

In certain embodiments of the invention, combination treatment with the agent configured to inhibit and/or diminish COX-2 expression, and/or PGE2 expression, and/or EP4 expression in bone related cells and a course of a drug known for treating metabolic disorders characterized with increased COX-2 expression and/or PGE2 expression and/or EP4 expression in bone related cells.

In addition to administering the agent is administered as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The agents (e.g., COX-2 inhibitors) and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example demonstrates that sensory denervation reduces osteoblastic bone formation.

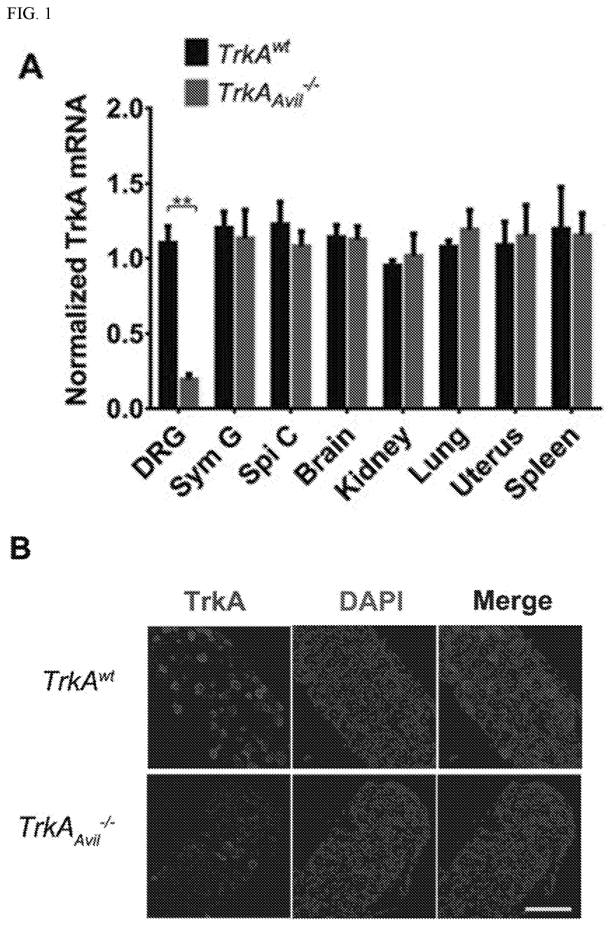

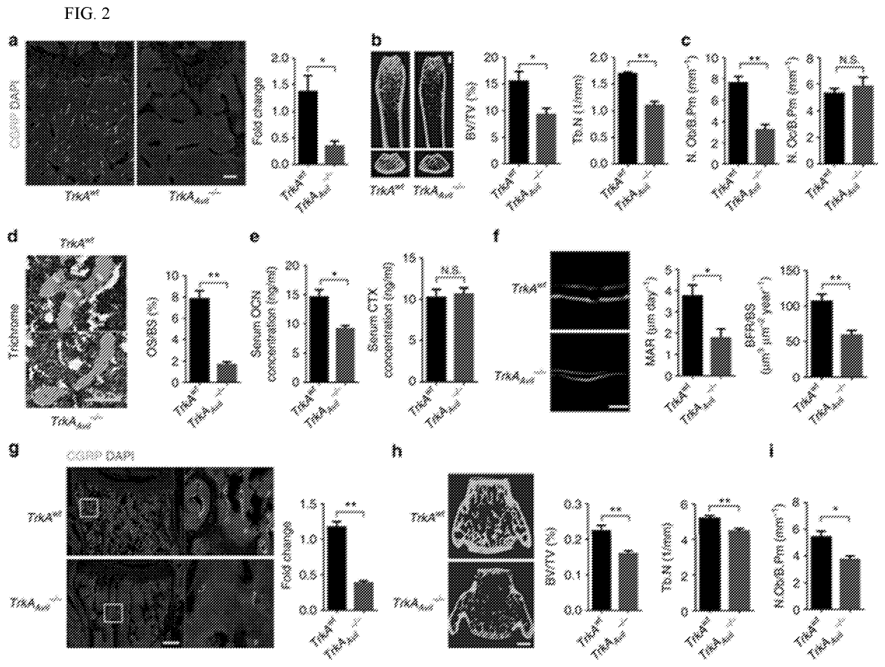

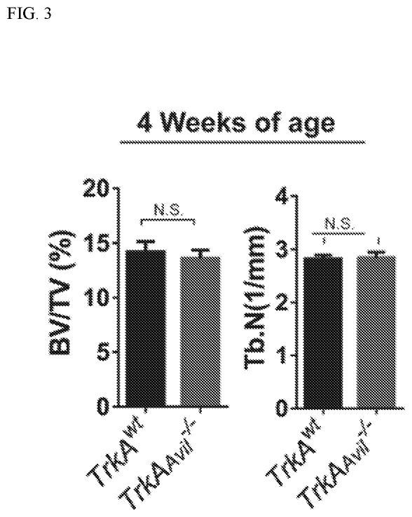

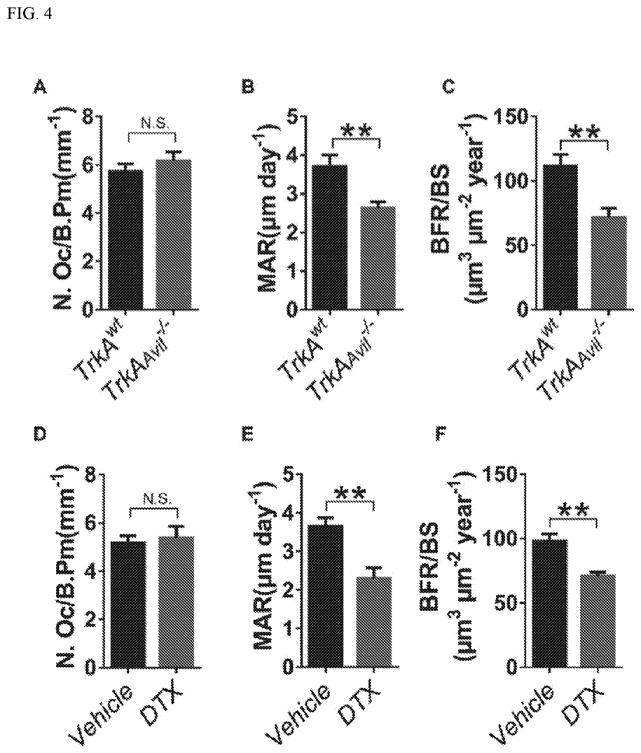

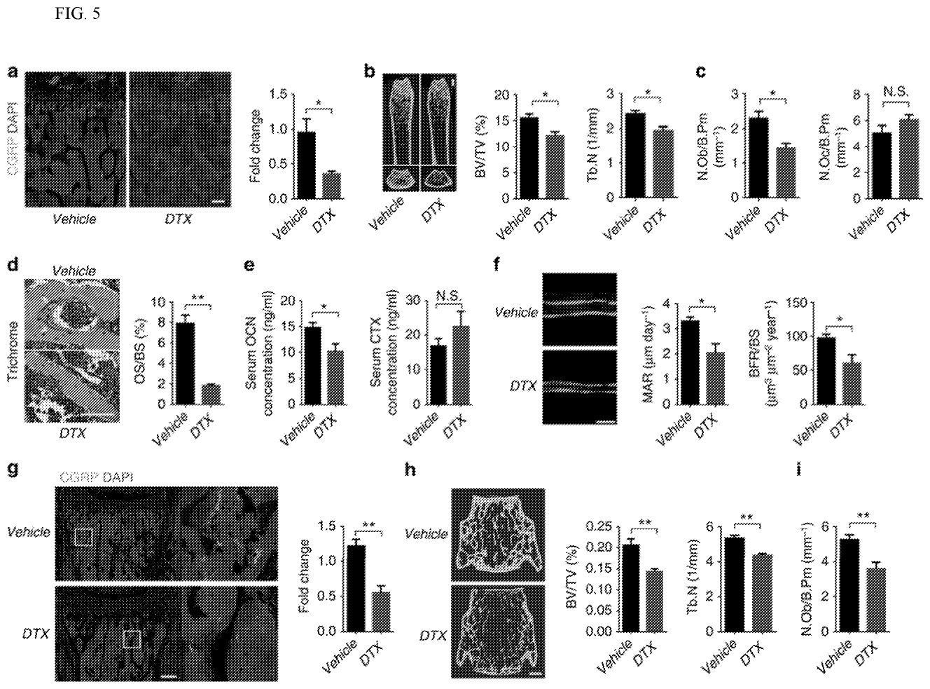

To investigate the effect of sensory nerve in bone, experiments were conducted that created a sensory denervation mouse model (TrkA_{Avil}^{-/-}) by crossing sensory nerve-specific cre (Advillin-cre) mice with nerve growth factor (NGF) receptor TrkA floxed (TrkA^{wt}) mice. Quantitative polymerase chain reaction (qPCR) and immunofluorescent staining of TrkA in the dorsal root ganglion (DRG) neurons and the other tissues isolated from the TrkA_{Avil}^{-/-} mice validated the knockout efficiency and specificity of the TrkA gene in the TrkA_{Avil}^{-/-} mice (FIG. 1). Furthermore, immunostaining of femur sections showed that most calcitonin gene-related peptide (CGRP)⁺ sensory nerve fibers were eliminated in the TrkA_{Avil}^{-/-} mice (FIG. 2A). Significant bone loss was observed in 12-week-old TrkA_{Avil}^{-/-} mice relative to their wild-type (WT) littermates in pCT analysis (FIG. 2B), while no significant bone volume decrease was found of 4-week-old age (FIG. 3), indicating an essential role of sensory nerve for bone homeostasis in adults. The number of osteocalcin⁺ osteoblasts was significantly lower in TrkA_{Avil}^{-/-} mice relative to their WT littermates; whereas, the number of tartaric acidic phosphatase (TRAP)⁺ osteoclasts was not different (FIG. 2C). Trichrome staining showed decreased osteoid in TrkA_{Avil}^{-/-} mice (FIG. 2D). Accordingly, the serum level of osteocalcin, a marker of osteoblastic bone formation, was significantly lower, and the level of osteoclast bone resorption marker carboxy-terminal collagen crosslinks (CTX) was not different in TrkA_{Avil}^{-/-} mice (FIG. 2E). Calcein double labeling confirmed the reduced bone formation and mineral apposition rate (FIG. 2F). Experiments were conducted that also evaluated the sensory innervations and bone architecture in the vertebrae of TrkA_{Avil}^{-/-} mice, and similar results were observed (FIG. 2G-I; FIG. 4A-C). These results indicate that sensory nerve regulates osteoblastic bone formation in adult mice.

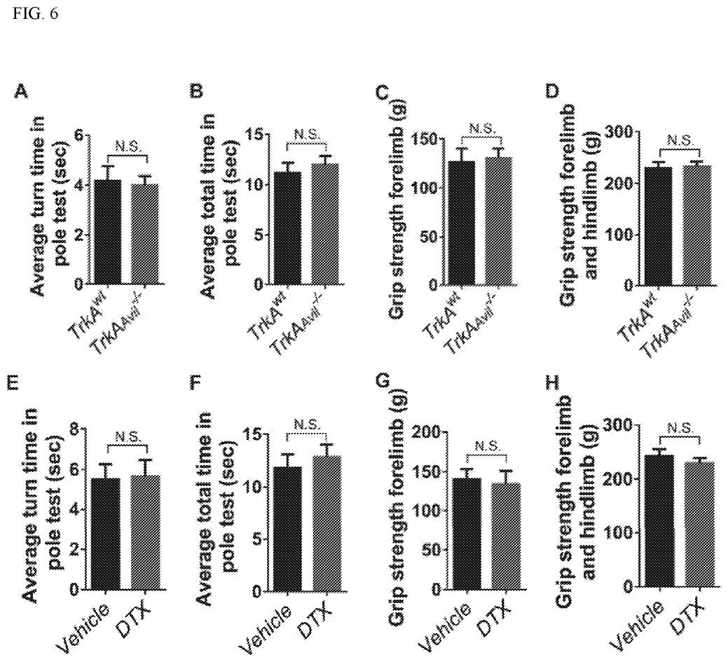

To examine whether sensory nerves maintain bone homeostasis through bone remodeling in adult mice, experiments were conducted that established inducible sensory denervation in iDTR_{Avil}^{+/-} mice by crossing Advillin-cre mice with iDTR^{wt} mice. Sensory denervation was effectively induced in adult iDTR_{Avil}^{fl/-} mice by injection of 1 ug per kg diphtheria toxin (DTX) three times a week for four weeks (FIG. 5A). Significant bone loss was observed using μCT (FIG. 5B). Similarly, the number of osteoblasts, amount of osteoid, and serum osteocalcin level were significantly decreased; whereas, the number of osteoclasts and serum CTX level were unchanged relative to the vehicle group (FIG. 5C, E). The calcein double-labeling experiment confirmed that bone formation and mineral apposition rate were reduced (FIG. 5F). Moreover, sensory innervations and bone mass also decreased in the vertebrae of iDTR_{Advil}^{fl/-} mice injected with DTX relative to the vehicle-treated mice (FIG. 5G, I:, FIG. 4D-E). Because neural changes other than those in the sensory nervous system can affect bone metabolism indirectly, pole tests and grip strength tests were performed with TrkA^{-/-} mice and iDTR_{Avil}^{fl/-} mice injected with DTX. No motor neural activity was influenced in these two mouse models (FIG. 6). Thus, sensory nerve regulates bone homeostasis through osteoblasts during bone remodeling.

### Example II

This example demonstrates that knockout of PGE2 receptor EP4 in sensory nerve induces bone loss.

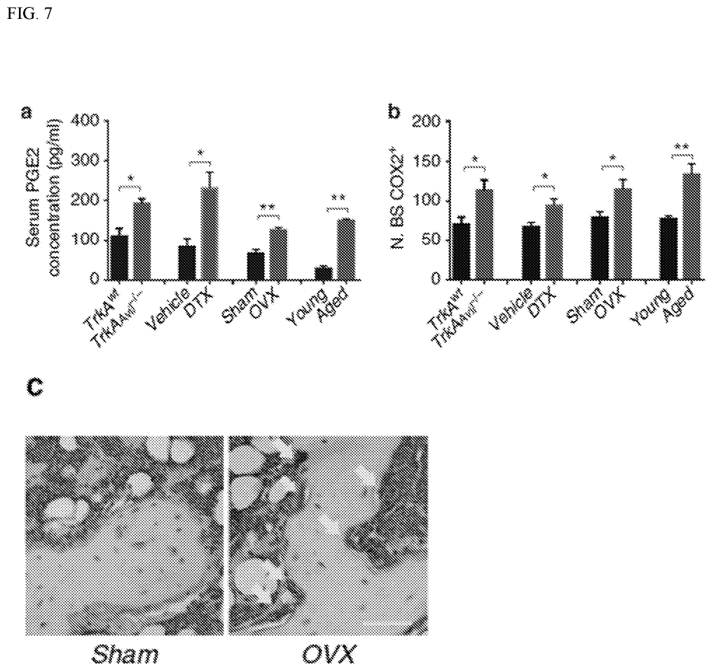

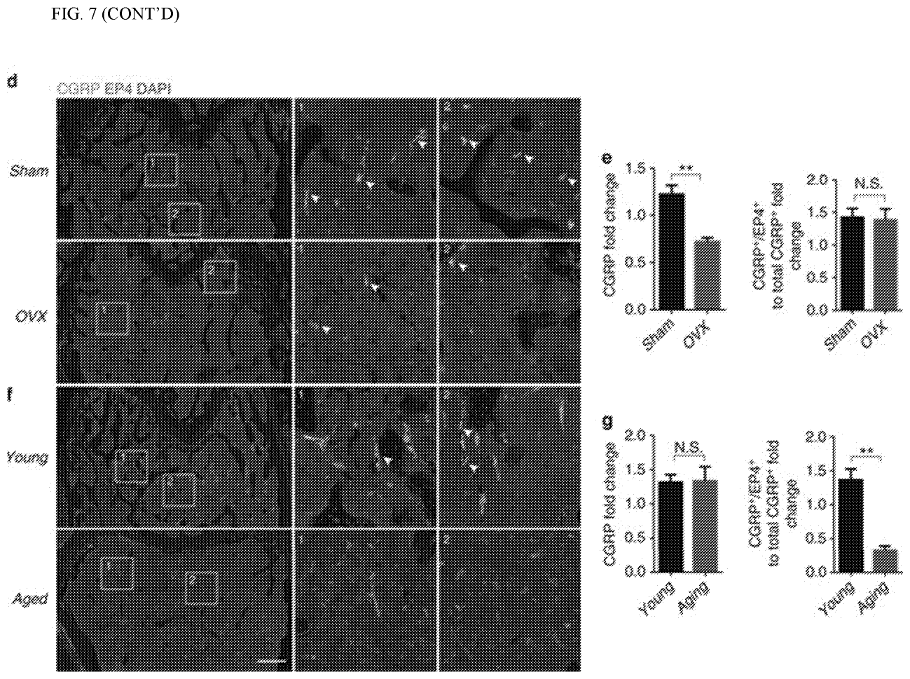

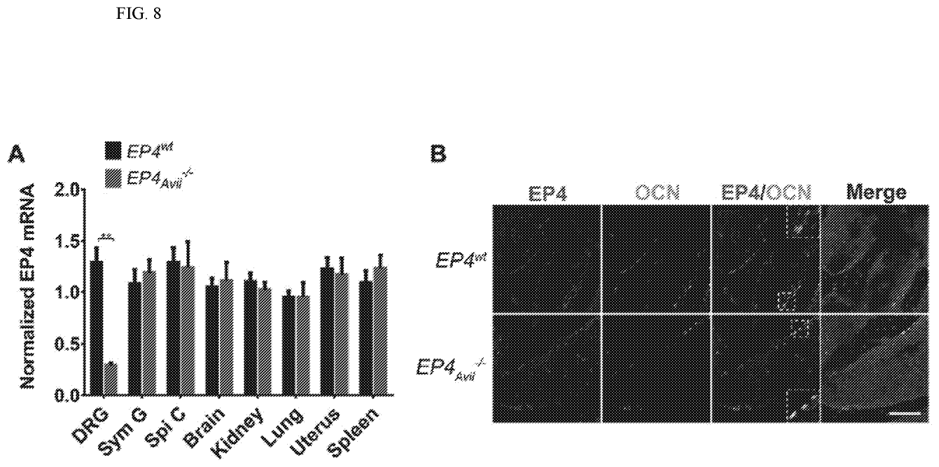

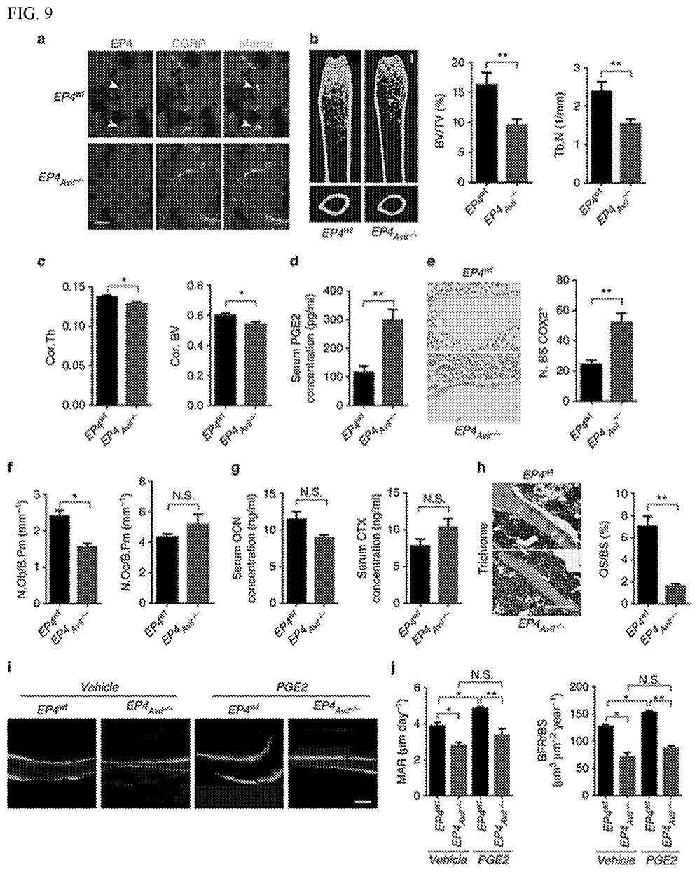

Because PGE2 is known to stimulate osteoblastic bone formation, experiments were conducted that measured PGE2 levels in the serum of both global and inducible sensory denervation mice. Interestingly, PGE2 levels increased significantly in all the denervation mouse models (FIG. 7A). The results prompted us to examine whether PGE2 mediates sensory nerve in regulation of osteoblast bone formation. It was found that bone density was negatively correlated with PGE2 levels, and that PGE2 levels increased in aged or the other osteoporotic mice (FIG. 7A). Immunohistochemical analysis also showed that expression of COX2 in femur osteoblasts, the PGE2 production-limiting enzyme, increased in the sensory denervation OVX and aged mice (FIG. 7B, C). As EP4 is the primary receptor of PGE2 for bone formation (see, Yoshida, K. et al. Proc. Natl. Acad. Sci. U. S. A. 99, 4580-4585), experiments were conducted that further co-immunostained of EP4 or CGRP in OVX and aged mice femurs. In OVX mice, a significant reduction of CGRP⁺ sensory fibers two weeks post OVX surgery was observed (FIG. 7D, E). Interestingly, loss of EP4 expression in the sensory fibers of aged mouse bone marrow with no significant decrease of the CGRP⁺ nerve fibers (FIG. 7F, G). Experiments were conducted that then induced ablation of EP4 in sensory nerves using adult EP4_{Avil}^{-/-} mice by crossing Advillin-cre mice with EP4^{wt} mice to validate of EP4 function in sensory nerves. qPCR analysis and immunostaining of EP4 confirmed that EP4 deletion was specifically in nerve (FIG. 8). Co-immunofluorescent staining of EP4 with CGRP showed that EP4 was expressed in bone sensory nerves, confirming that EP4 was efficiently deleted from sensory nerves in the bone marrow of EP4_{Avil}^{-/-} mice (FIG. 9A).

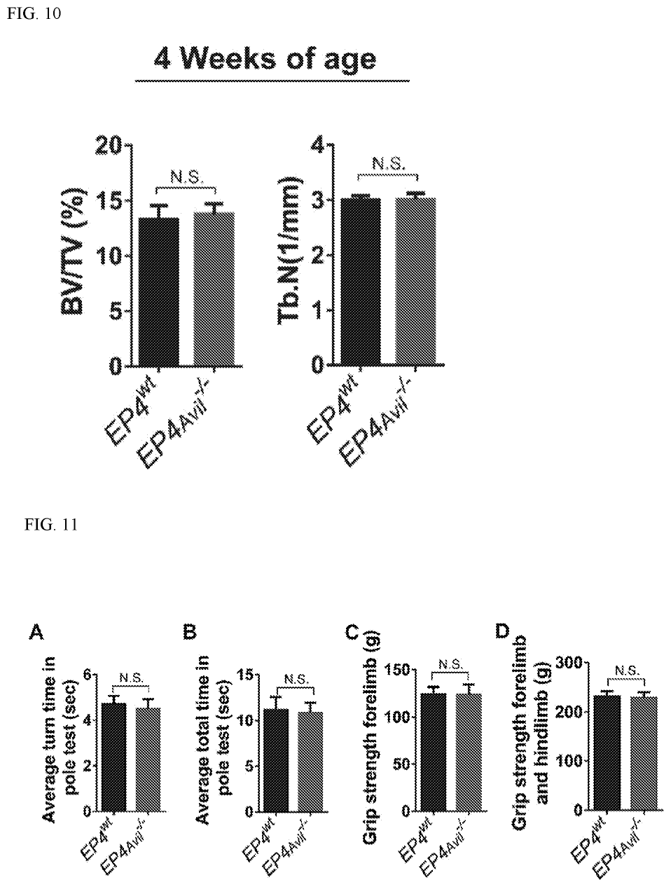

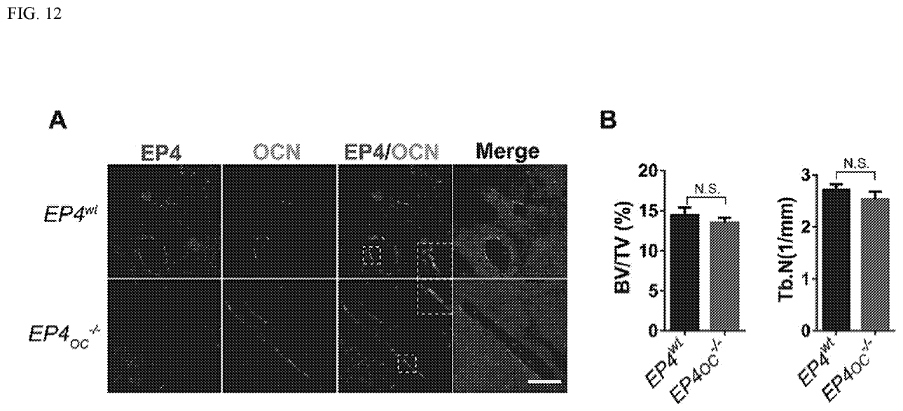

Both trabecular bone and cortical bone decreased significantly in 12-week EP4-ablated mice (FIG. 9B,C; FIG. 10). Pole tests and grip strength tests showed no changes in motor neural activity (FIG. 11). PGE2 levels in the serum and COX2 expression in osteoblasts increased (FIG. 9D, E), suggesting a compensatory increase of the ligand PGE2 in response to EP4 knockout in sensory nerves. Similar to the two sensory denervation models, in EP4_{Avil}^{-/-} mice, the number of osteoblasts decreased (but the serum osteocalcin level showed no statistical significance), with no changes in osteoclast number or bone degradation marker CTX (FIG. 9F, G). Importantly, PGE2 induced significant bone formation in wild-type mice was abolished in EP4_{Avil}^{-/-} mice, as demonstrated in trichrome and double-labeling experiments (FIG. 7H, J). As EP4 expression is also known to express in osteoblasts, osteoblastic cell-specific knockout EP4 mice were generated by crossing EP4^{wt} mice with osteocalcin-cre (OC-cre) mice (FIG. 12A). However, pCT analysis did not reveal significant bone volume change in EP4_{OC}^{-/-} mice (FIG. 12B). These results show that PGE2-induced osteoblastic bone formation is signaled through EP4 in the sensory nerves.

### Example III

This example demonstrates that PGE2 mediates sensory nerve-induced osteogenesis.

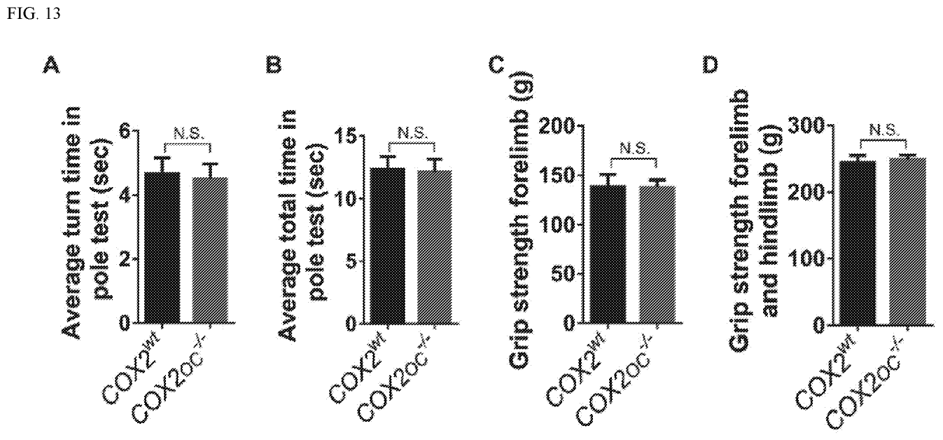

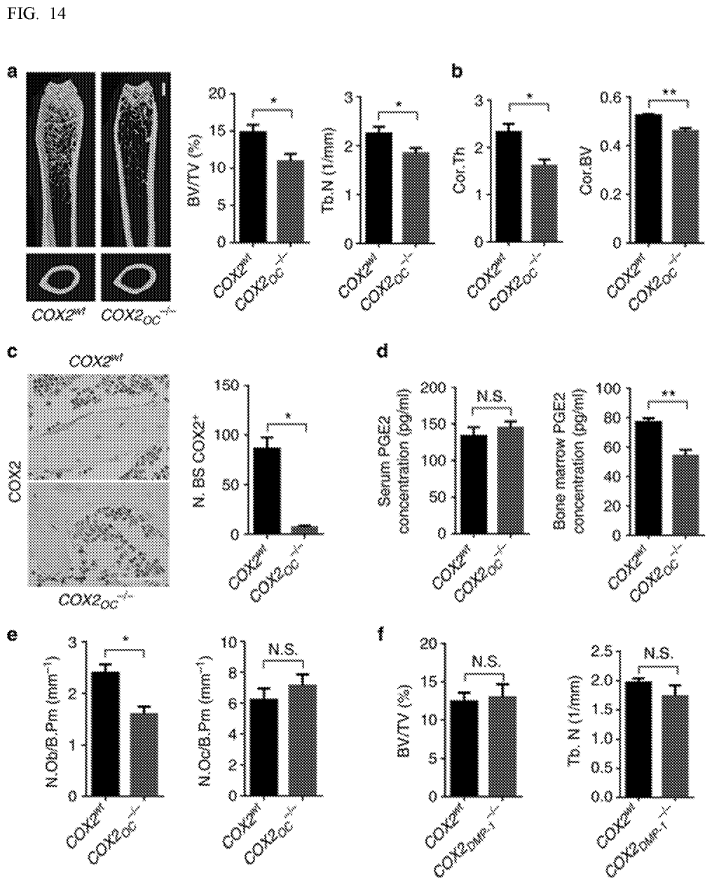

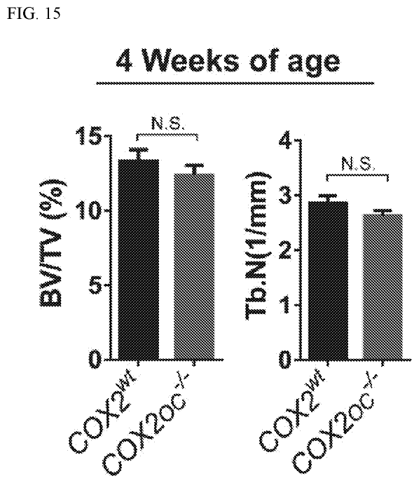

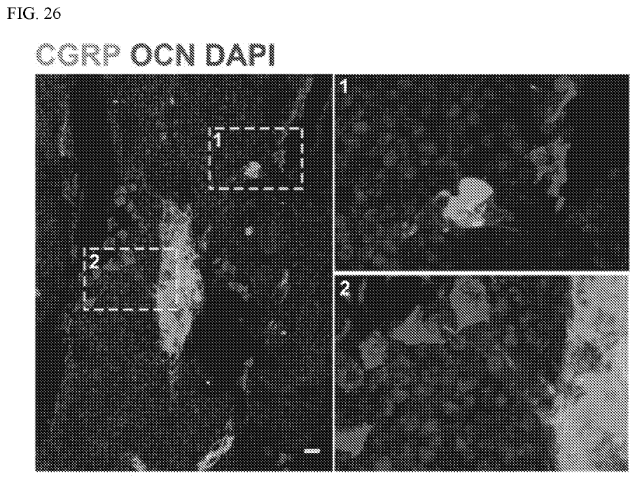

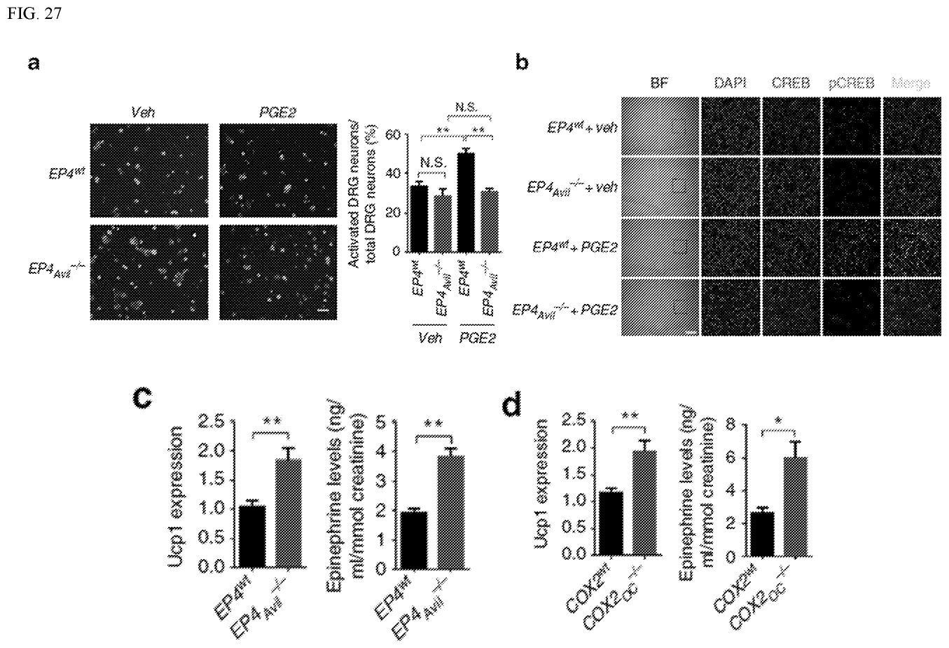

To examine whether PGE2 is secreted primarily by osteoblastic cells for sensory nerve regulation of bone, experiments were conducted that further generated conditional knockout COX2 mice in the osteoblastic cells (COX2_{OC}^{-/-}) by crossing COX2^{wt} mice with OC-cre mice to eliminate PGE2 secretion by osteoblastic cells. Pole tests and grip strength tests showed no effect on motor activity, indicating that knockout of COX2 did not affect global neural activity (FIG. 13). As in EP4 knockout mice, trabecular and cortical bone decreased while body weight remained unchanged over time in COX2_{OC}^{-/-} mice relative to their COX2^{wt} littermates (FIG. 14A, B; FIG. 15). COX2 staining of the mouse femurs confirmed the expression of COX2 and its ablation in osteoblasts (FIG. 14C). Interestingly, CGRP⁺ nerve fibers were located in the active bone remodeling areas with Ocn⁺ osteoblasts in co-immunostaining (FIG. 26). This suggests that PGE2 secreted by osteoblastic cells in active bone remodeling sites is essential because the bone marrow PGE2, instead of the serum PGE2 levels, was significantly different between these two groups of mice (FIG. 14D). Thus, PGE2 secreted by osteoblastic cells in active bone remodeling sites mediates sensory nerve-stimulated bone formation. Again, the number of osteoblasts and serum osteocalcin level decreased significantly, whereas TRAP⁺ osteoclast number and serum CTX level were unchanged (FIG. 14E).

Experiments were conducted that also deleted COX2 in osteocytes embedded in the bone matrix from terminal differentiation of osteoblasts to examine PGE2 in the osteoblastic bone-forming microenvironment essential for sensory nerve-induced osteogenesis. Crossing DMP 1 -cre mice with COX2^{wt} mice generates COX2_{Dmp1}^{-/-} mice to eliminate PGE2 secretion by osteocytes. Interestingly, bone phenotype was unchanged in COX2_{DMP1}^{-/-} mice relative to their COX2^{wt} littermates (FIG. 14F). Taken together, these results show that PGE2 in the active bone-forming microenvironment, largely secreted by osteoblasts, mediates sensory nerve-regulated osteoblastic bone formation.

### Example IV

This example demonstrates that PGE2 induces hypothalamic CREB signaling through peripheral sensory nerves for osteogenesis.

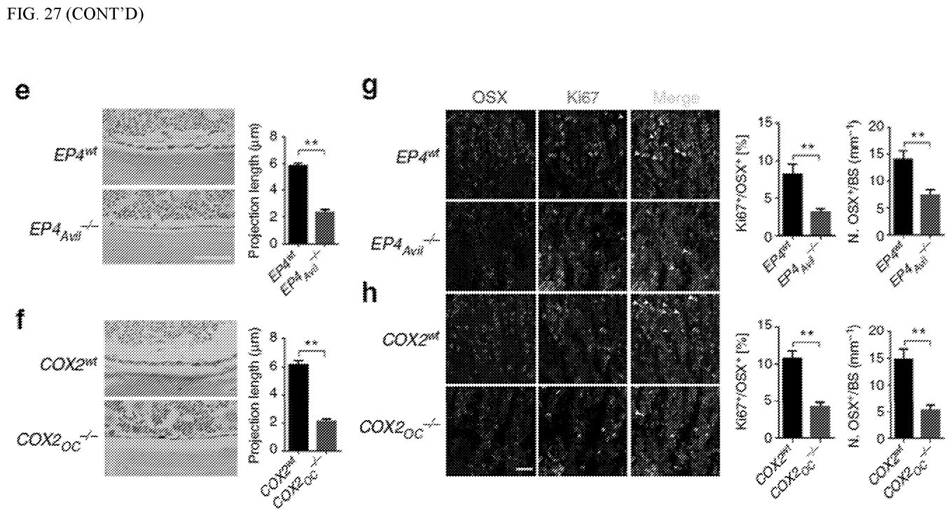

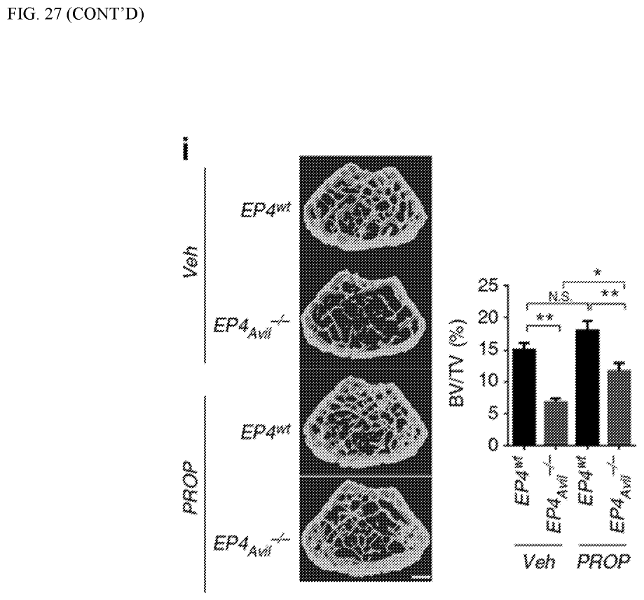

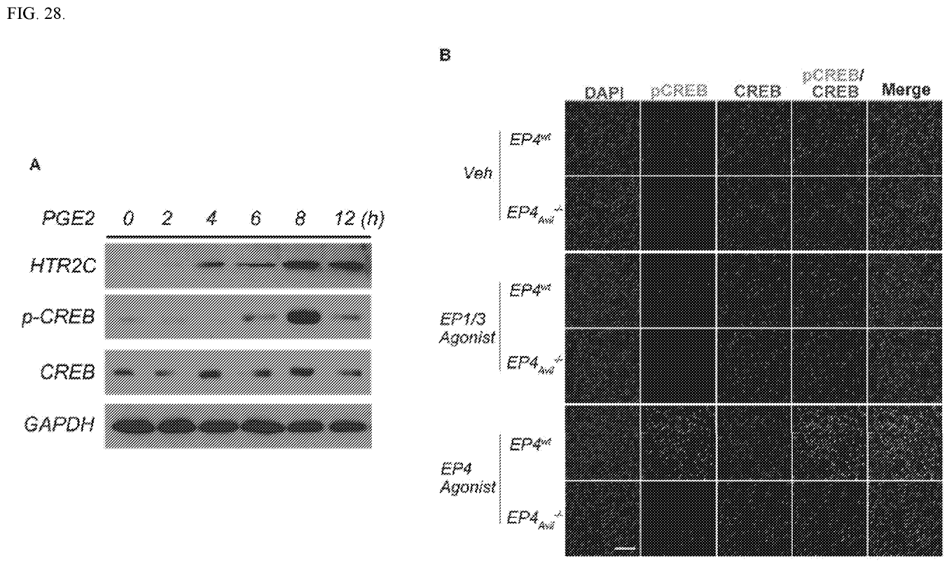

CREB signaling in the hypothalamus is crucial for the regulation of skeletal homeostasis (see, Oury, F. et al. Genes Dev. 24, 2330-2342, doi:10.1101/gad.1977210 (2010)). To examine whether PGE2 could activate EP4 in sensory nerves through the ventromedial nucleus of the hypothalamus (VMH), experiments were conducted that examined the effect of PGE2 on DRG neurons and the phosphorylation of CREB in the VMH of EP4_{Avil}^{-/-} mice. Calcium imaging showed more illuminated DRG neurons in those pre-treated with PGE2 relative to vehicle-treated neurons, whereas DRG neuron activation was reduced significantly in EP4_{Avil}^{-/-} mice with or without PGE2 pre-treatment (FIG. 27A). Western blot analysis of the hypothalamus showed that phosphorylation of CREB increased gradually and peaked at 6 h after injection (FIG. 28A). Immunostaining of VMH sections showed that phosphorylation of CREB decreased significantly in EP4_{Avil}^{-/-} mice relative to their WT littermates (FIG. 27B). PGE2 was then injected into EP4_{Avil}^{-/-} mice and their WT littermates to further test whether the central regulation is sensory nerve-dependent. Immunostaining of hypothalamus sections showed CREB phosphorylation increased significantly in mouse VMH 6 h after injection, whereas, PGE2-induced CREB phosphorylation in VMH was abolished in EP4_{Avil}^{-/-} mice (FIG. 27B). Then, EP1/3 and EP4 agonists were injected to examine whether EP4 receptor is responsible for PGE2-induced CREB signaling in VMH. EP1/3 agonist did not increase phosphorylation of CREB in VMH, whereas EP4 agonist significantly increased pCREB level relative to vehicle-treated mice (FIG. 28B), indicating that EP4 in sensory nerves is specific for PGE2-induced CREB phosphorylation in VMH.

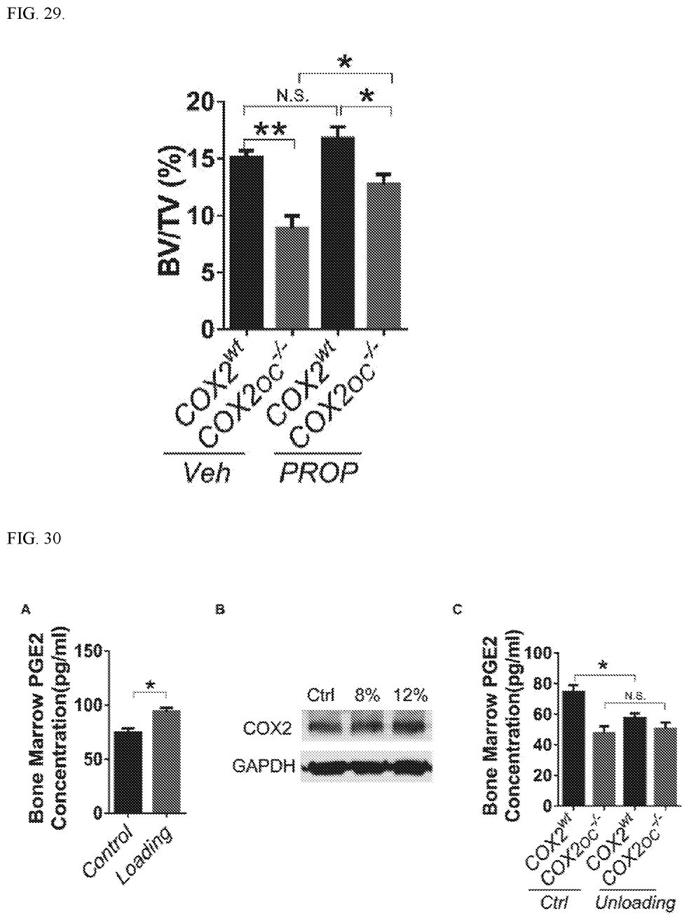

The activation of CREB signaling in the hypothalamus has been shown to suppress sympathetic tone (see, Oury, F. et al. Genes Dev. 24, 2330-2342, doi:10.1101/gad.1977210 (2010); Ortuno, M. J. et al. Nat. Med. 22, 1170-1179, doi:10.1038/nm.4166 (2016)). Indeed, uncoupling protein 1 gene (UCP 1) expression in adipose tissue and epinephrine concentrations in urine increased significantly in the EP4_{Avil}^{-/-} mice and COX2_{OC}^{-/9-} mice relative to their WT littermates, indicating higher sympathetic tone in these two mouse models (FIG. 27C, D). Immunostaining of the femur sections showed small, flattened osteoblasts on the bone surface (FIG. 27E, F) and reduced Ki67 expression in osterix⁺ cells in both COX2_{OC}^{-/-} and EP4_{Avil}^{-/-} mice, indicating that increased sympathetic tone suppress osteoblastic activity (FIG. 27G, H). To confirm that the increased sympathetic activity leads to bone loss, experiments were conducted that injected propranolol, a β2-adrenergic antagonist, into EP4_{Avil}^{-/-} mice and COX2_{OC}^{-/-} mice. Propranolol partially rescued bone phenotype of these two knockout mice (FIG. 271; FIG. 29). These results indicate that sympathetic tone regulates osteoblast differentiation through EP4 activation of sensory nerve.

Experiments were conducted that also tested if PGE2 secretion is regulated by mechanical loading as mechanical loading has been shown to regulate bone homeostasis through central regulation of sympathetic tone (see, Hino, K. et al. J. Cell Biochem. 99, 845-852 (2006); Kondo, H. et al. Unloading induces osteoblastic cell suppression and osteoclastic cell activation to lead to bone loss via sympathetic nervous system. J. Biol. Chem. 280, 30192-30200 (2005); Kondo, H. et al. J. Biol. Chem. 280, 30192-30200 (2005)). Mechanical loading was applied to C57B/L6 mice and bone marrow PGE2 levels were measured. The result showed that PGE2 levels significantly increased in the loading group compared with the control group (FIG. 30A). COX2 expression in osteoblasts was examined in osteoblastic MC3T3-E1 cells cultured on high-extension silicon rubber dishes with applied compression force to mimic mechanical loads on bone. Western blot analysis showed that COX2 expression increased when the compression force applied (FIG. 30B). In addition, bone marrow PGE2 levels in COX2_{OC}^{-/-} mice and their littermates with unloading by tail suspension were measured. The results showed that bone marrow PGE2 significantly decreased in wild-type mice in tail suspension conditions while no change was observed in COX2_{OC}^{-/-} mice with (FIG. 30C). These results suggest that osteoblasts secrete PGE2 secretion in responsible to mechanical loading.

### Example V

This example demonstrates that PGE2 promotes skeletal regeneration through sensory nerves.

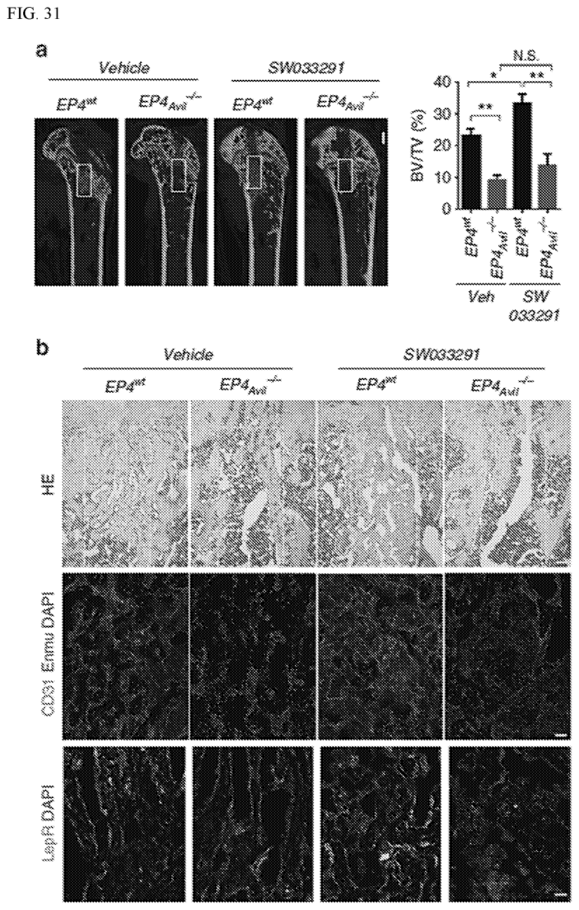

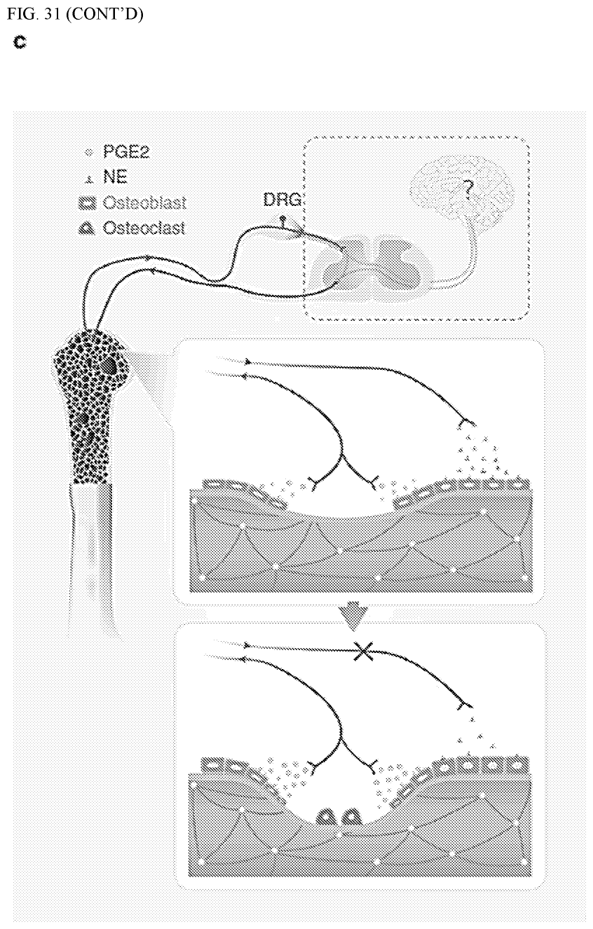

PGE2 has been reported recently to potentiate regeneration of multiple tissues (see, Zhang, Y. et al. Science 348, aaa2340, doi:10.1126/science.aaa2340 (2015)). To investigate whether PGE2 induces tissue regeneration through sensory nerves, experiments were conducted that assessed whether PGE2 induces bone regeneration. 15-PGDH inhibitor SW033291 was injected into EP4_{Avil}^{-/-} and EP^{wt} mice that had undergone surgical ablation of trabecular bone to examine the effects of an increase in local PGE2 on bone regeneration. Elevation of local PGE2 boosted trabecular bone regeneration significantly in EP4^{wt} mice injected with SW033291 relative to vehicle-treated controls, as shown by μCT (FIG. 31A) and hematoxylin-eosin (HE) staining (FIG. 31B). However, the regeneration of trabecular bone by SW033291 was obstructed in EP4_{Avil}^{-/-} mice (FIG. 31A, B). As known that CD31^{hi} Endomucin^{hi} type H vessel couples with active new bone formation (see, Kusumbe, A. P., Ramasamy, S. K. & Adams, R. H. Nature 507,323-328 (2014); Xie, H. et al. Nat. Med. 20, 1270-1278 (2014)), experiments were conducted that further evaluated its expression and found that the type H vessel growth was significantly increased in the regeneration area of EP4^{wt} mice treated with SW033291. However, type H vessels were almost undetectable in EP4_{Avil}^{-/-} mice with or without injection of SW033291 (FIG. 31B). Thus, PGE2 stimulates bone regeneration through sensory nerves.

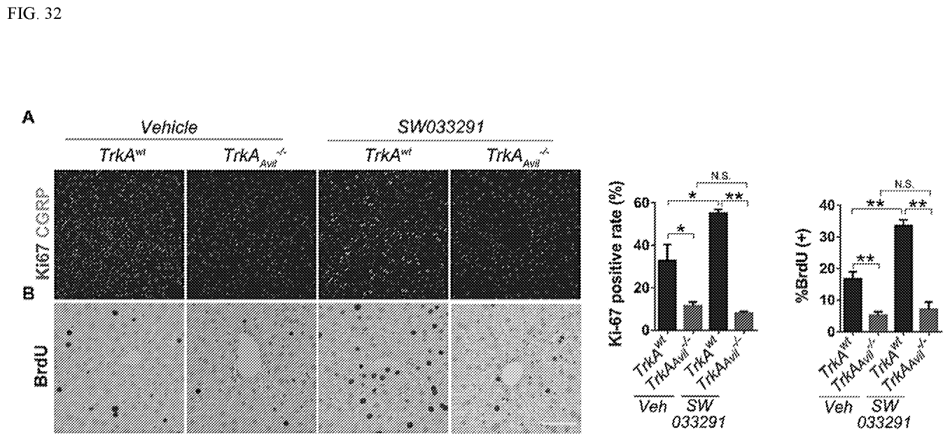

To further investigate whether PGE2 induces regeneration by sensory nerves of tissues other than bone, experiments were conducted that performed partial hepatectomy in TrkA_{Avil}^{-/-} mice and their WT littermates treated with SW033291 or vehicle. BrdU and Ki67 staining of liver sections with partial hepatectomy showed that the regeneration rate decreased significantly in TrkA_{Avil}^{-/-} mice treated with SW033291 relative to their WT littermates (FIG. 32). Staining of CGRP in the regeneration areas confirmed that sensory innervation in the liver was significantly reduced in TrkA_{Avil}^{-/-} mice. These results show that PGE2 induces bone regeneration dependent on sensory nerves, and that sensory nerves are likely involved in regeneration of various tissues.

### Example VI

This example provides the materials and methods for Examples I-V.

### Mice and in Vivo Treatment

The iDTR^{fl/fl} and Dentin matrix acidic phosphoprotein 1-Cre (DMP1-Cre) mice were purchased from the Jackson Laboratory. The Advillin-Cre (Avil-Cre) mouse strain was kindly provided by Xingzhong Dong (The Johns Hopkins University). The Osteocalcin-Cre (OC-cre) mice were obtained from Thomas J. Clemens (The Johns Hopkins University). The TrkAP^{fl/fl} mice were obtained from David D. Ginty (Harvard Medical School). The COX2^{fl/fl} mice were provided by Harvey Herschman (University of California, Los Angeles). The EP4^{fl/fl} mice were obtained from Brian L. Kelsall (National Institutes of Health). Heterozygous male Avil-Cre mice (female Avil-Cre mice were not used to breed in case for the leakage of TrkA protein into the eggs) were crossed with a TrkA^{fl/fl}, EP4^{fl/fl}, or iDTR^{fl/fl} mouse. The offspring were intercrossed to generate the following genotypes: wild type (referred as WT in the text), Avil-Cre (Cre recombinase expressed driven by Advillin promoter), TrkA^{fl/fl} (mice homozygous for TrkA flox allele are referred to as TrkA^{wt} in the text), EP4^{fl/fl} (referred to as EP4^{wt} in the text), iDTR^{fl/fl}, Avil-Cres:EP4^{fl/fl} (conditional deletion of EP4 receptor in Advillin lineage cells, referred to as EP4_{Avil}^{-/-} in the text), Avil-Cre:: TrkA^{fl/fl} (referred to as TrkA_{Avil}^{-/-} in the text), and Avil-Cre::iDTR^{fl/+} mice (referred to as iDTR_{Avil}^{+/-} in the text). Heterozygous OC-Cre or DMP 1-Cre mice were crossed with a COX2^{fl/fl} mouse; the offspring were intercrossed to generate the following genotypes: WT, OC-Cre, DMP1-Cre, COX2^{fl/fl} (referred to as COX2^{wt} in the text), OC-Cre:: COX2^{fl/fl} (referred to as COX2_{OC}^{-/-} in the text), and DMP1-Cre:: COX2 ^{fl/fl} (referred to as COX2_{Dmp1}^{-/-} in the text) mice. Heterozygous OC-cre mice were crossed with a EP4^{fl/fl} mouse, the offspring were intercrossed to generated the following genotypes: WT (referred as EP4^{fl/fl}) and OC-cre:: EP4^{fl/fl} (conditional deletion of EP4 receptor in osteocalcin lineage cells, referred to as EP4_{OC}^{-/-} in the text). The genotypes of the mice were measured by PCR analyses of genomic DNA, which was extracted from mouse tails within the following primers: Avil-Cre: forward: CCCTGTTCACTGTGAGTAGG (SEQ ID NO:1), Reverse: GCGATCCCTGAACATGTCCATC (SEQ ID NO:2), WT:AGTATCTGGTAGGTGCTTCCAG (SEQ ID NO:3); OC-Cre: forward: CAAATAGCCCTGGCAGATTC (SEQ ID NO:4), Reverse: TGATACAAGGGACATCTTCC (SEQ ID NO:5); DMP1-Cre forward: TTGCCTTTCTCTCCACAGGT (SEQ ID NO:6), Reverse: CATGTCCATCAGGTTCTTGC (SEQ ID NO:7); EP4 loxP allele forward: TCTGTGAAGCGAGTCCTTAGGCT (SEQ ID NO:8), Reverse: CGCACTCTCTCTCTCCCAAGGAA (SEQ ID NO: 9);COX2 loxP allele forward: AATTACTGCTGAAGCCCACC (SEQ ID NO:10), Reverse: GAATCTCCTAGAACTGACTGG (SEQ ID NO:11); TrkA loxP allele forward: AACAGTTTTGAGCATTTTCTATTGTTT (SEQ ID NO:12), Reverse: CAAAGAAAACAGAAGAAAAATAATAC (SEQ ID NO:13); iDTR loxP allele forward: GCGAAGAGTTTGTCCTCAACC (SEQ ID NO:14), Reverse: AAAGTCGCTCTGAGTTGTTAT (SEQ ID NO:15). 8 to 12-week-old C57BL/6 female mice (Jackson Lab) were anesthetized and underwent bilateral OVX or a sham operation from back approach. The aged mice (12 months old) were purchased from The Jackson

Laboratory. All animals were maintained at the animal facility of The Johns Hopkins University School of Medicine. All animal experimental protocols were complied with all relevant ethical regulations and approved by the Animal Care and Use Committee of The Johns Hopkins University, Baltimore, Md., USA. Whole blood samples were obtained by cardiac puncture immediately after euthanasia. Serum was collected by centrifuge at 200×g for 15 min and stored at −80° C. before analyses. Femurs, tibias, and urine of the mice were also collected.

The drugs and compounds used in this study are as follows: diphtheria toxin (DTX, Sigma-Aldrich, D0564); PGE2 (Cayman Chemical, 14010); EP1/3 agonist (Cayman Chemical, 14810); EP4 agonist (Cayman Chemical, 10580); propranolol (Sigma-Aldrich, 1576005); norepinephrine (Sigma-Aldrich, A7257); and SW033291 (Selleck, S7900). Dosages and time courses are noted in the corresponding text and figure legends.

### Behavioral Analysis

Pole tests and grip strength tests were performed to evaluate motor neural activity changes in TrkA_{Avil}^{-/-}, EP4_{Avil}^{+/-}, and COX2_{OC}^{-/-} mice. All tests were performed between 10:00 and 16:00 during the lights on cycle. For the pole test, a 9-mm-diameter metric as 0.76-m metal rod wrapped with bandage gauze was used as the pole. The time for a mouse to turn and the total time for it to reach the base of the pole was recorded. Before the test, the mice were trained for three consecutive days, and each training session consisted of three test trials. For grip strength, neuromuscular strength was measured as maximum holding force generated by the mice (Biosed, USA). Mice were placed to grasp a metal grid with their forelimbs or hindlimbs. The tail was pulled gently, and the maximum holding force was recorded by the force transducer when the mice released their grasp on the grid. The peak holding strength was recorded digitally and displayed in grams.

### μCT Analyses

The femurs were harvested from mice, and the soft tissue around the bone was removed, followed by fixation overnight using 4% paraformaldehyde. μCT analyses were performed by using a high-resolution μCT scanner (SkyScan, 1174). The voltage of the scanning procedure was 65 kv with a 153-μA current. The resolution was set to 8.7 μm per pixel. Reconstruction software (NRecon, v1.6, SkyScan), data analysis software (CTAn, v1.9, SkyScan), and 3D model visualization software (CTVol, v2.0, SkyScan) were used to analyze the diaphyseal cortical bone and the metaphyseal trabecular bone parameters of the femurs. Experiments were conducted that created cross-sectional images of the femur to perform 2D analyses of the cortical bone and 3D analyses of the trabecular bone. The region of interest (ROI) of the trabecular bone was drawn beginning from 5% of the femur length proximal to the distal metaphyseal growth plate and extending proximally for another 5% of the total femur length. The trabecular bone volume fraction (BV/TV), trabecular thickness (Tb. Th), trabecular number (Tb. N), and trabecular separation (Tb. Sp) were collected from the 3D analyses data and used to represent the trabecular bone parameters. The cortical bone ROI was drawn beginning from 20% of femur length proximal to distal metaphyseal growth plate and extending proximally to another 10% of the total femur length. The cortical thickness (Ct. Th), periosteal perimeter (Ps. Pm), and endosteal perimeter (Es. Pm) were collected from the 2D analyses data and used to represent the cortical bone parameters.

### Immunohistochemistry, Immunofluorescence, and Histomorphometry

The femurs were collected and fixed in 4% paraformaldehyde overnight and decalcified by using 10% EDTA (pH, 7.4) (Amresco, 0105) for 21 days. The samples were then dehydrated with 30% sucrose for 24 h and embedded in paraffin or optimal cutting temperature compound (Sakura Finetek). 4-μm-thick coronal-oriented sections of the femur were prepared for hematoxylin and eosin staining. The femurs were fixed for 4 h with 4% paraformaldehyde at 4° C. and then decalcified at 4° C. using 0.5M EDTA (pH, 7.4) for 24 h with constant shaking. The samples were dehydrated in 20% sucrose and 2% polyvinylpyrrolidone (PVP) solution for 24 h and embedded in 8% gelatin (Sigma-Aldrich, G1890) in the presence of 20% sucrose and 2% PVP. Forty-μm-thick coronal-oriented sections of the femurs were obtained. For brain section preparation, the whole brain was collected from euthanized mice and fixed with 4% paraformaldehyde for 30 mins. Then, the tissue was dehydrated with 20% sucrose for 24 h and sectioned.

Immunostaining was performed using standard protocol. Briefly, the sections were incubated with primary antibodies to mouse osterix (Abcam, ab22552, 1:600), osteocalcin (Takara Bio, M173, 1:200), CD31 (Abcam, ab28326, 1:50), endomucin (Santa Cruz, V.7C7, 1:50), Ki67 (Abcam, ab16667, 1:100), CGRP (Abcam, ab81887, 1:100), COX2 (Abcam, ab15191, 1:100), EP4 (Abcam, ab92763, 1:10), CREB (Cell Signaling Technology, 9197, 1:100), p-CREB (Abcam, ab32096, 1:100), NF200 (Millipore, AB5539, 1:500), TrkA (R& D systems, AF1056, 1:1000) and IB4 (Thermo Fisher Scientific, 121411) overnight at 4° C. A horseradish peroxidase-streptavidin detection kit (Dako) was used in immunohistochemical procedures to detect immuno-activity, followed by counter staining with hematoxylin (Dako, S3309). Fluorescence-conjugated secondary antibodies were used in immunofluorescent procedures to detect fluorescent signals after counter staining with DAPI (Vector, H-1200). A Zeiss LSM 780 confocal microscope or an Olympus BX51 microscope was used for sample image capturing. A BrdU staining kit (Thermo Fisher Scientific, 8800-6599-45) was used to perform the BrdU immunostaining procedure. Quantitative histomorphometric analysis was performed by using OsteoMeasure XP Software (OsteoMetric) in a blinded fashion.

A double-labeling procedure was performed to measure dynamic bone formation. Briefly, 0.1% calcein (Sigma-Aldrich, C0875) was injected in phosphate-buffered saline at a concentration of 10mg per kg into the mice subcutaneously 7 days and 1 day before sacrifice. The double-labeling images of undecalcified bone slices were captured using a fluorescence microscope. Trabecular bone formation in four randomly selected visual fields was analyzed in the distal metaphyseal area of the femur.

Quantitative Real-Time Polymerase Reaction Chain (qPCR)

Total RNA was purified from cells in culture or tissues using TRIzol (Invitrogen, 15596026), following the manufacturer's protocol. Experiments were conducted that performed qPCR using the Taq SYBR Green Power PCR Master Mix (Invitrogen, A25777) on a CFX Connect instrument (Bio-Rad); Gapdh amplification was used as an internal control. Dissociation curves analysis was performed for every experiment. Sequences of the primers used for each gene are as listed: EP4 forward: CGGTTCCGAGACAGCAAA (SEQ ID NO:16), Reverse: CGGTTCGATCTAGGAATGG (SEQ ID NO:17). UCP 1 forward: CTTTGCCTCACTCAGGATTGG (SEQ ID NO:18), Reverse: ACTGCCACACCTCCAGTCATT (SEQ ID NO:19). TrkA: AGAGTGGCCTCCGCTTTGT (SEQ ID NO:20), Reverse: CGCATTGGAGGACAGATTCA (SEQ ID NO:21). Gapdh forward: ATGTGTCCGTCGTGGATCTGA (SEQ ID NO:22), Reverse: ATGCCTGCTTCACCACCTTCTT (SEQ ID NO:23).

### Bone Marrow Supernatant Collection

Mice were euthanized, cut two ends of the tibia, and centrifuged the samples for 15 min at 800×g at 4° C. to obtain bone marrow supernatants, which were stored at −80° C. until ELISA.

### ELISA and Western Blot Testing

PGE2 concentrations in the serum and bone marrow were determined by PGE2 ELISA kit (Cayman Chemical, 514010) according to the manufacturer's protocol. Mice serum was collected as described above. Experiments were conducted that also performed osteocalcin and CTX ELISA of serum using a mouse osteocalcin enzyme immunoassay kit (Biomedical Technologies, BT-470) and a RatLaps enzyme immunoassay kit (Immunodiagnostic Systems, ACO6F1).

Western blot analysis was conducted on the basis of the protein lysates from the hypothalamus of mice or cultured cell line. The lysates were centrifuged; the supernatants were collected and separated by SDS-PAGE PAGE (sodium dodecyl sulfate-polyacrylamide gel electrophoresis) and then blotted on the polyvinylidene fluoride membrane (Bio-Rad Laboratories). Specific antibodies were applied for incubation, and the proteins were detected by using an enhanced chemiluminescence kit (Amersham Bioscience, RNP2108). The antibodies used for western blotting were HTR2C (Abcam, ab197776, 1:500), CREB (Cell Signaling Technology, 9197, 1:500), p-CREB (Abcam, ab32096, 1:500), COX2 (Abcam, ab15191, 1:1000), and GAPDH (Cell Signaling Technology, 5174, 1:1000).

### DRG Culture and Calcium Imaging

DRGs from the L2-L5 spinal levels of 4-week-old mice were isolated in cold DMEM/F12 medium (Invitrogen, 11039-021) and then treated with collagenase type A (Roche, 10103578001) at 37° C. After trituration and centrifugation, cells were resuspended and plated on glass coverslips coated with ploy-D-lysine and laminin. The cells were then cultured in an incubator at 37° C. Primary isolated DRG neurons were loaded with Fura-2-acetomethoxyl ester (Molecular Probes) for 45 min in the dark at room temperature. The cells were imaged at 340 and 380 nm after immersion in calcium-free buffer for 1 min.

### In Vitro and in Vivo Mechanical Loading Assays

In vitro-mechanical stretching assay: Osteoblastic MC3T3 cells (stored in our lab) were plated on the home-made high-extension silicone rubber dishes with 4% and 12% morphological change for 24 h and starved for 4 h. The high-extension silicone rubber dishes were fixed on the home-made mechanical stretching machine and compress the cells with different force reflected as 4% and 12% morphology change of the dishes. After 24 h, the cells lysate was harvested for western blot assays.

In vivo-treadmill assay: C57BL/6 mice were trained for 5 days on treadmill with the protocol of 8 m/min, 5° uphill for 10 min per day. Then, the protocol of 14 m/min, 14° uphill for 20 min per day for formal tests was followed. After 2 weeks, serum and bone marrow were harvested for ELISA assays.

### Bone and Liver Regeneration Models

Mice underwent general anesthesia. The bone regeneration model was established as described below. A longitudinal incision was made on each knee to expose the femoral condyle by patella dislocation. Then, a hole was made at the intercondylar notch of the femur by using a dental drill. A 0.6-mm-diameter Kirschner wire was placed from the proximal end of the femur to confirm marrow ablation by radiography. The dislocated patella was reposed, and the skin was sutured after removal of the Kirschner wire. Bone samples were harvested 7 days after bone marrow ablation, as described above.

Partial hepatectomy was used for the liver regeneration model. 10- to 12-week-old TrkA_{Avil}^{F592A} male mice were anesthetized, as described above. A partial (two-thirds) hepatectomy was performed by resecting the median and left lateral hepatic lobes⁵⁴. The remnant livers were harvested after mice sacrifice. SW033291 was dissolved in a vehicle of 10% ethanol, 5% Cremophor EL, and 85% dextrose 5% in water.

### Statistical Analysis

All data analyses were performed using SPSS, version 15.0, software (IBM Corp.). Data are presented as means±standard error of mean (SEM). For comparisons between two groups, two-tailed Student t-tests was used. For comparisons among multiple groups, one-way ANOVA was used. All inclusion/exclusion criteria were pre-established, and no samples or animals were excluded from the analysis. No statistical method was used to predetermine the sample size. The experiments were randomized. The investigators were not blinded to allocation during experiments and outcome assessment. All relevant data are available from the authors.

### Example VII

This example describes aberrant subchondral bone formation in spontaneous OA mice.

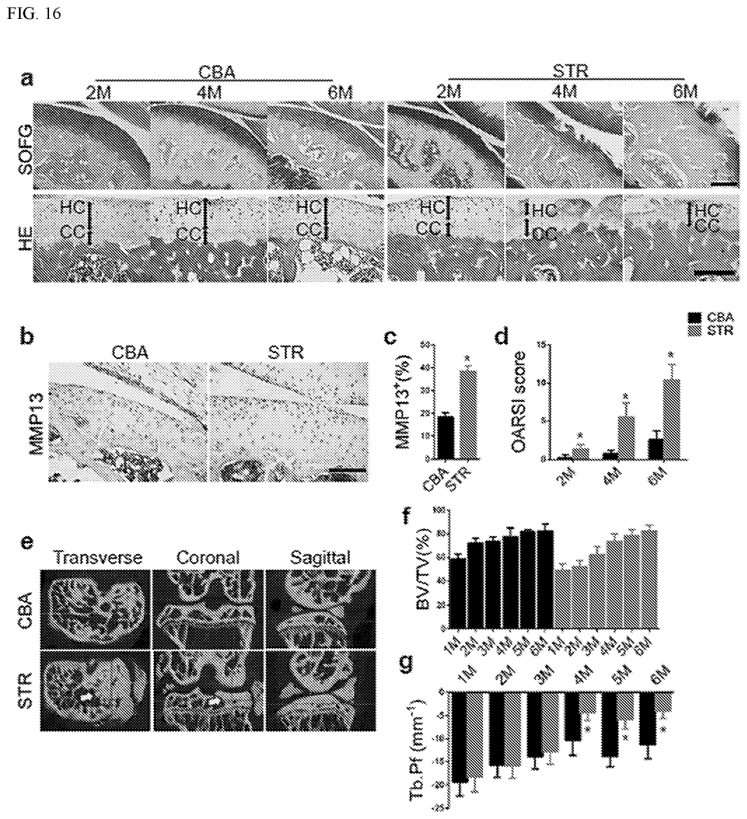

To examine the pathogenesis of spontaneous OA, experiments were conducted that sectioned knee joints of STR/Ort mice at different ages for immunostaining analysis. The results showed that significant proteoglycan loss occurred at the deeper zone of cartilage adjacent to the subchondral bone from 4 months of age and became aggravated over time relative to the age-matched control CBA mice (FIG. 16a). The tidemark moved up because of the decreased thickness of the hyaline cartilage zone and the increased calcified cartilage layer shown by hematoxylin and eosin staining (FIG. 16a). The percentage of MMP13⁺ chondrocytes increased significantly in 4-month-old STR/Ort mice (FIG. lb, c), suggesting the degeneration of articular cartilage in STR/Ort mice. Osteoarthritis Research Society International (OARSI) scores (see, Pritzker, K. P., et al. Osteoarthritis and cartilage 14, 13-29 (2006)) showed that significant articular cartilage degeneration started from 4 months of age in STR/Ort mice and progressed gradually relative to the age-matched CBA mice (FIG. 16d).

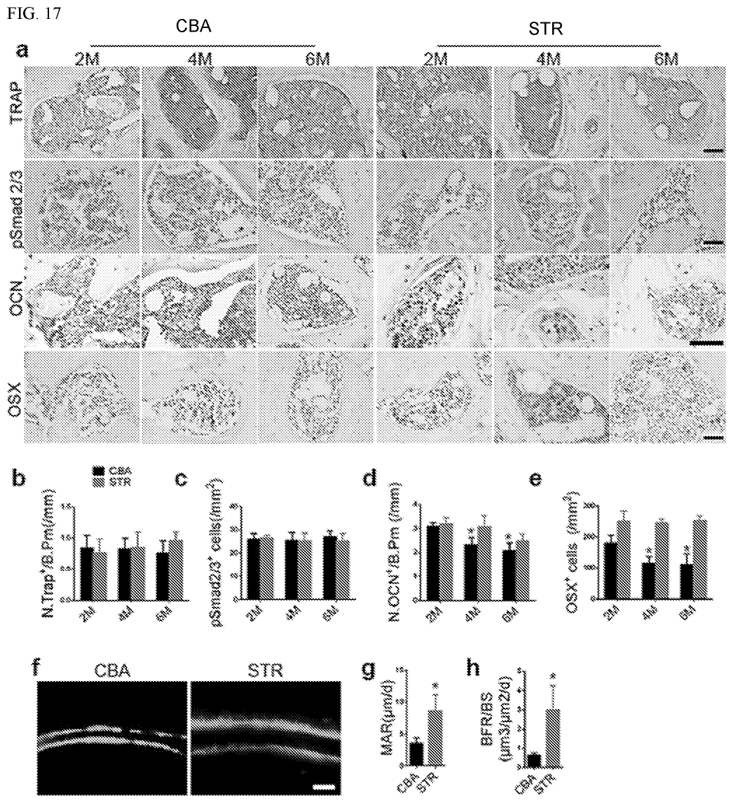

Experiments next examined tibial subchondral bone using 3-dimensional microcomputed tomography (μCT) analysis because aberrant subchondral bone formation causes OA (see, Zhen, G., et al. Nature medicine 19, 704-712 (2013)). The sectional, coronal, and sagittal views of tibial subchondral bone showed an uneven distribution of bone mass in STR/Ort mice relative to CBA controls, indicating uncoupled bone formation (FIG. 16e). The ratio of subchondral bone volume (BV)/tissue volume (TV) increased each month by more than 10% in STR/Ort mice, whereas the BV/TV in CBA controls remained relatively steady after puberty (FIG. 160. Furthermore, the increase in trabecular pattern factor (Tb.Pf.) indicates a decrease in subchondral bone connectivity in STR/Ort mice (FIG. 16g). Increases in osteoclasts and transforming growth factor-β (TGF-β) activity in subchondral bone induce traumatic OA in anterior cruciate ligament transection mice (see, Zhen, G., et al. Nature medicine 19, 704-712 (2013)). However, tartrate-resistant acid phosphatase (TRAP)⁺ osteoclasts and pSmad2/3⁺ cells represent for TGF-β signaling showed no changes in STR/Ort mice of different ages, similar to CBA mice (FIG. 17a-c). Interestingly, immunostaining showed that the numbers of osteocalcin (OCN)⁺ osteoblasts and osterix⁺ osteoprogenitors increased significantly in the subchondral bone marrow from 4 months of age in STR/Ort mice relative to CBA mice (FIG. 17a,d,e), indicating osteoblastic bone formation. The increase in bone formation rate and mineral apposition rate in calcein double-labeled bone confirmed subchondral bone formation (FIG. 17f-h). Therefore, the pathogenesis of aberrant subchondral bone formation in spontaneous OA does not involved in elevated osteoclasts and transforming growth factor-0 in traumatic OA.

### Example VIII

This example demonstrates elevated COX-2 activity in the subchondral bone of spontaneous OA.

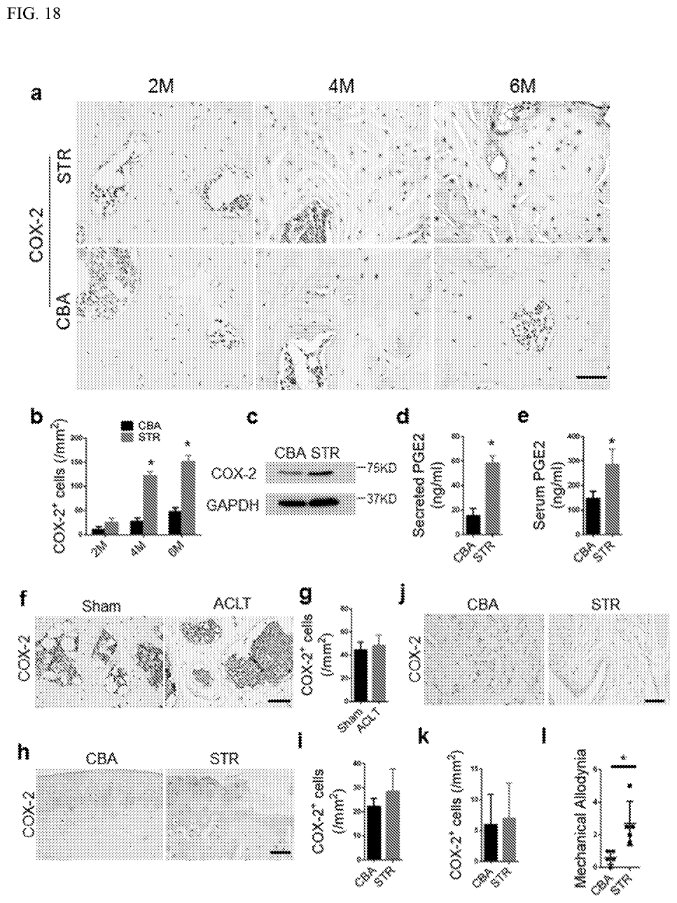

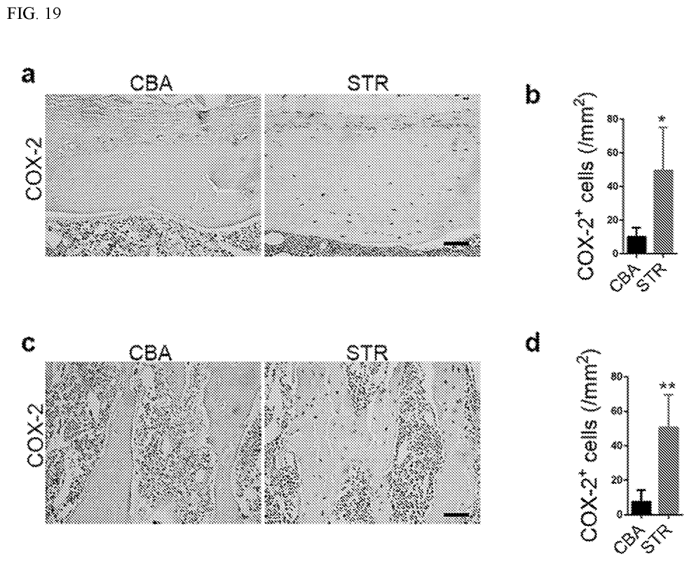

Because PGE2 stimulates bone formation without osteoclast bone remodeling (see, Blackwell, K. A., Raisz, L. G. & Pilbeam, C. C. Trends in endocrinology and metabolism: TEM 21, 294-301 (2010)), experiments examined whether COX-2 is elevated in subchondral bone formation in STR/Ort mice. Immunostaining showed a significant increase in COX-2⁺ osteocytes in the subchondral bone starting at 4 months of age (FIG. 18a, b), COX-2 expression in non-subchondral bone osteocytes were also increased (FIG. 19a-d). COX-2 protein levels in subchondral bone osteocytes were elevated in STR/Ort mice (FIG. 18c), and PGE2 secreted by osteocytes of STR/Ort mice increased significantly (FIG. 18d). The serum PGE2 level of 4-month-old STR/Ort mice was twice as high as that of CBA controls (FIG. 18e). There was no increase in COX-2 expression in the subchondral bone of traumatic OA mice (FIG. 18f, g), nor in the articular cartilage chondrocytes or synovial cells of STR/Ort mice compared with controls at 4 months of age (FIG. 18h-k). A high level of COX-2 is associated with joint pain, as shown in the Von Frey test, with a higher response rate in STR/Ort mice (FIG. 181). Collectively, these results indicate that genetically elevated COX-2 expression in the subchondral bone is associated with spontaneous OA but not traumatic OA of mechanically unstable joints.

### Example IX

This example demonstrates elevated COX-2 activity in the subchondral bone of genetically modified RA and both human OA and RA.

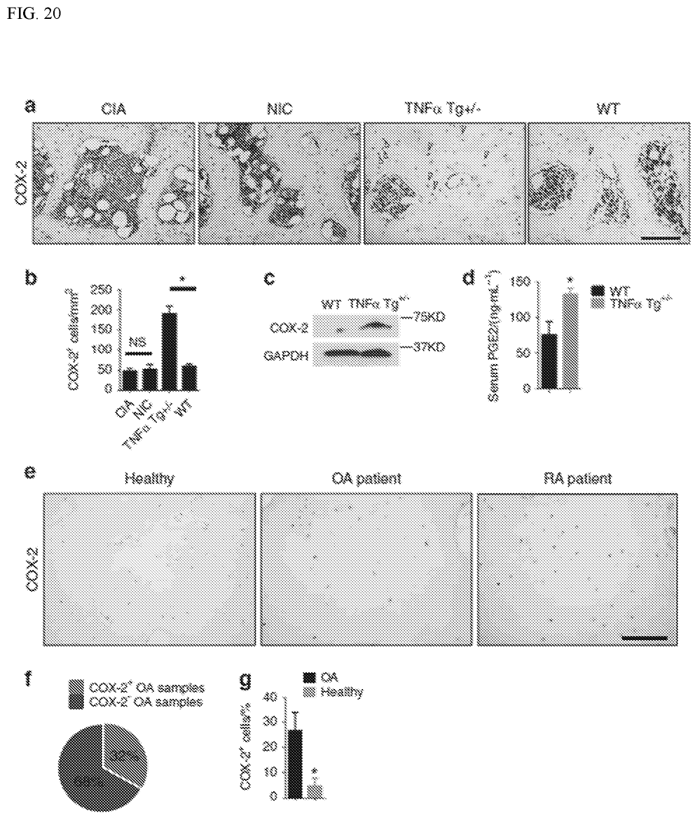

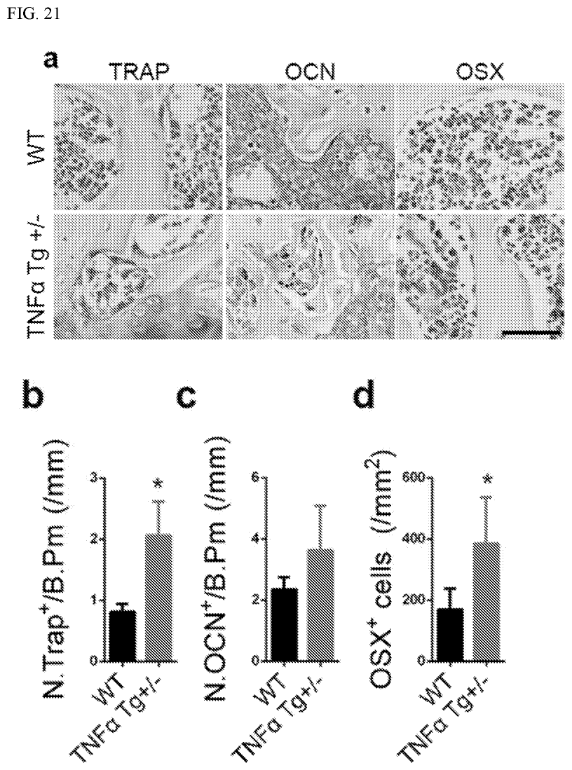

Experiments also examined COX-2 levels in the subchondral bone of 2 different RA mouse models. Interestingly, COX-2 levels were increased in TNF-α transgenic RA mice (TNF-α Tg^{+/-}), but not in type II collagen-induced RA mice (CIA) compared with controls (FIG. 20a, b). Western blot analysis confirmed higher levels of COX-2 expression in the subchondral bone of TNF-α transgenic mice (FIG. 20c). As expected, the serum PGE2 level also increased (FIG. 20d), as did the numbers of osterix⁺ osteoprogenitors, and TRAP⁺ osteoclasts in the subchondral bone of TNF-α transgenic mice (FIG. 21a, b and d). The OCN⁺ osteoblasts in subchonral bone of TNF-α transgenic mice showed no difference related to WT controls (FIG. 21a,c). Moreover, experiments evaluated the expression of COX-2 in the knee joint subchondral bone of human OA and RA patients. Approximately one-third of the 43 human OA knee samples, and all knee samples from 9 RA patients, showed elevated COX-2 expression relative to the healthy controls (FIG. 20e, f). The percentage of COX-2 positive cells was as high as 26% in these knee samples with upregulated COX-2 expression (FIG. 20g). The elevated COX-2 in spontaneous OA, genetically modified mouse RA, and human OA and RA specimens reveals COX-2 as a pathological process of both OA and RA.

### Example X

This example demonstrates that inhibition of COX-2 or conditional knockout of COX-2 in osteocytes attenuates progression of RA.

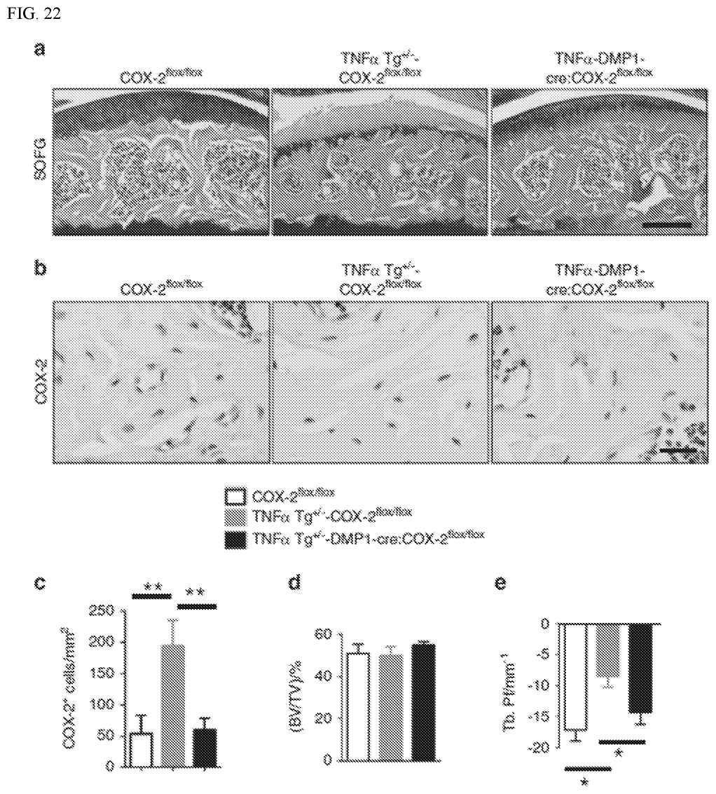

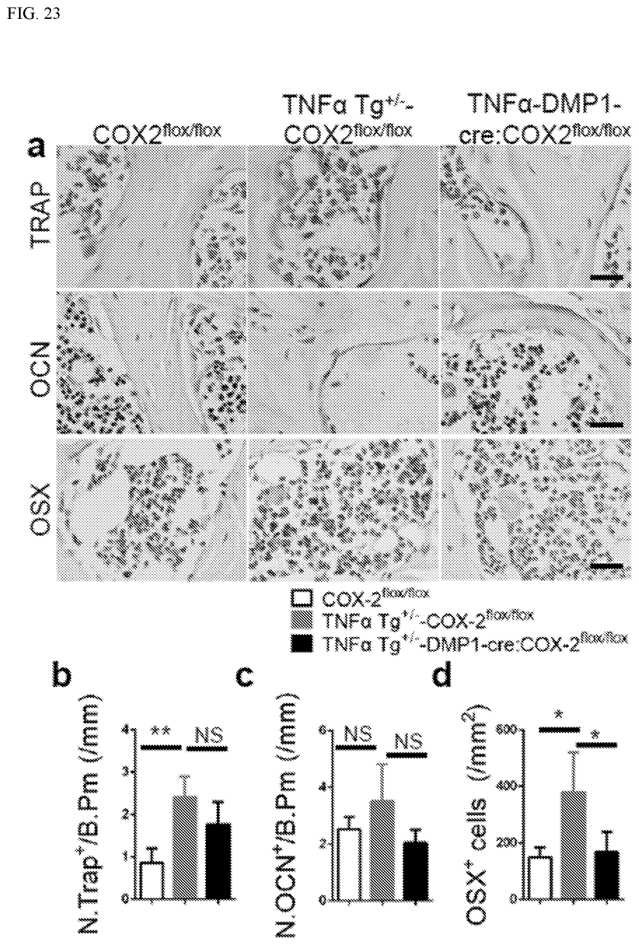

Dentin matrix protein 1 (DMP1) is a matrix protein, expressed in odontoblasts, preosteocytes, and osteocytes. DMP1-Cre transgenes are widely used to target osteocytes (see, Lu, Y., et al. Journal of dental research 86, 320-325 (2007); Kalajzic, I., et al. Bone 54, 296-306 (2013)). To specific knock out COX-2 in osteocytes in TNF-α transgenic mice, experiments crossed DMP1-Cre mice with TNF-α transgenic mice, and their progeny DMP1-Cre::TNF-α transgenic mice were further crossed with COX-2^{flox/flox} mice. Articular cartilage degeneration was attenuated in COX-2^{-/-} RA mice (TNF-α DMP1-Cre:: COX-2^{-/-}) relative to the TNF-α COX-2^{flox/flox} mice (FIG. 22a). As expected, the COX-2⁺ osteocytes decreased significantly (FIG. 22b, c), and the numbers of osteocancin⁺ osteoblasts and TRAP⁺ osteoclasts did not change significantly compared with controls (FIG. 23a-c), but the numbers of osterix⁺ osteoprogenitors of COX-2^{-/-} RA mice decreased relative to controls (FIG. 23a, d). Subchondral bone quality improved significantly, as evidenced by increased connectivity with no BV change (FIG. 22d, e).

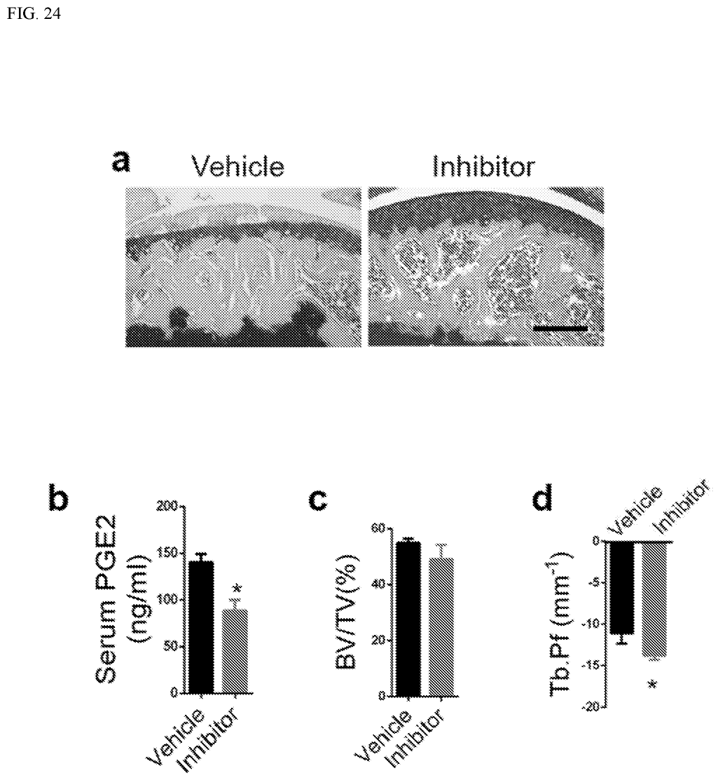

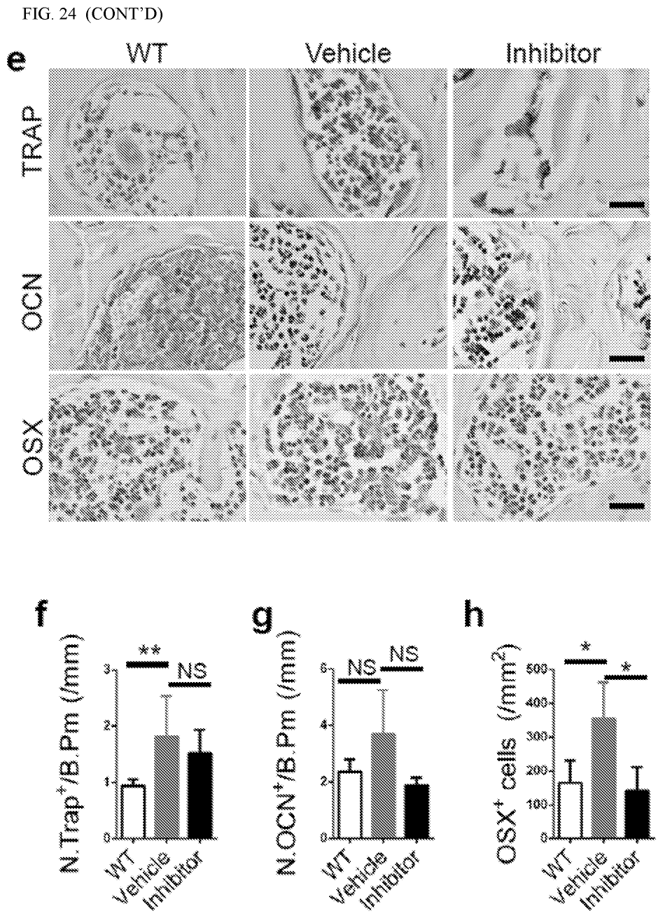

To further validate whether genetically elevated COX-2 expression caused arthritis, experiments were conducted that gavage fed COX-2 inhibitor celecoxib to TNF-α transgenic mice at a dose of 8 mg/kg daily for 4 weeks. The serum PGE2 level decreased significantly with the administration of COX-2 inhibitor (FIG. 24b). Importantly, degeneration of articular cartilage was ameliorated (FIG. 24a), and the numbers of osterix⁺ osteoprogenitors in the subchondral bone marrow decreased with COX-2 inhibitor administration (FIG. 24e,h). However, the numbers of osteocalcin+osteoblasts and TRAP⁺ osteoclasts did not change significantly in inhibitor-treated mice (FIG. 24e-g). Subchondral bone quality improved significantly, as evidenced by increased connectivity with no BV change (FIG. 24c, d).

Thus, the fact that inhibition or knockout of COX-2 attenuated RA validates that COX-2 is a heritable factor causing RA.

### Example XI

This example demonstrates that inhibition of COX-2 attenuates spontaneous OA.

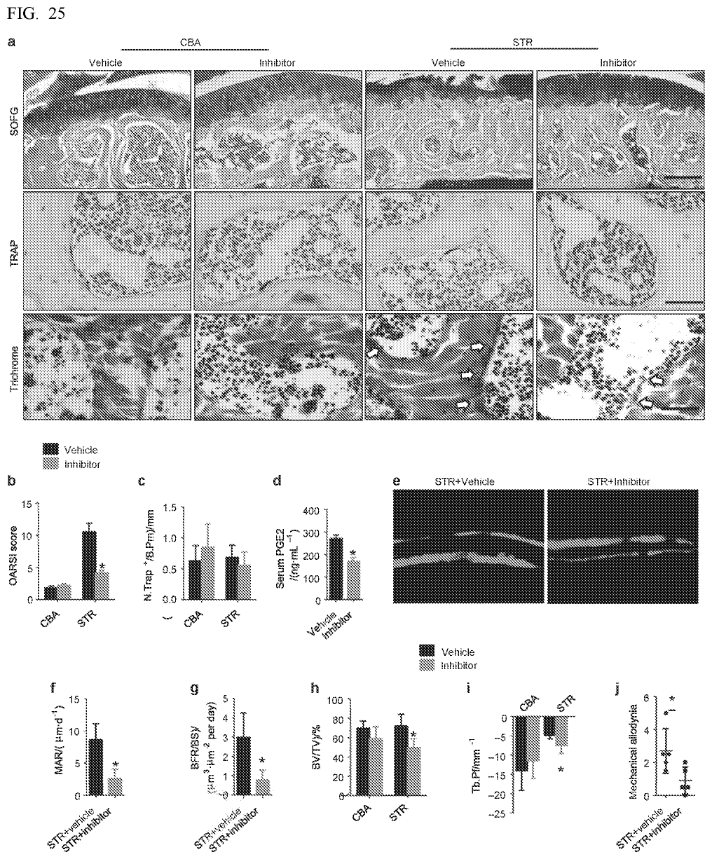

Experiments were conducted that examined whether inhibition of COX-2 also attenuates spontaneous OA. COX-2 inhibitor celecoxib was gavage fed at different dosages once a day for 4 weeks. Proteoglycan loss and calcification of articular cartilage were effectively attenuated by 8 mg/kg of COX-2 inhibitor in STR/Ort mice relative to controls, as indicated by safranin 0 and fast green staining (FIG. 25a); the OARSI score for articular cartilage also improved significantly (FIG. 25b). The serum PGE2 level decreased significantly in STR/Ort mice treated with COX-2 inhibitor, but the number of TRAP⁺ osteoclasts did not change (FIG. 25a, c, d). Subchondral bone osteoid formation decreased significantly on the bone surface with COX-2 inhibitor as indicated by trichrome staining (FIG. 25a). Calcein double-labeling confirmed that subchondral bone formation rate and mineral apposition rate were decreased in STR/Ort mice treated with COX-2 inhibitor (FIG. 25e-g). Subchondral bone trabeculae connectivity and microarchitecture improved significantly (FIG. 25h, i). Interestingly, Von Frey testing showed that pain sensitivity decreased after administration of COX-2 inhibitor in spontaneous OA mice (FIG. 25j), indicating that a decrease in PGE2 reduces pain. In parallel, CBA mice with the same genetic background as STR/Ort mice were treated with COX-2 inhibitor and exhibited no significant changes in subchondral bone or articular cartilage (FIG. 25a-d). Taken together, genetically elevated COX-2 in subchondral bone causes both spontaneous OA and RA.

### Example XII

This example describes the materials and methods for Examples VII through XI.

### Human Subjects

Human subchondral bone samples were obtained from 43 patients with OA and 9 patients with RA undergoing knee joint replacement or open reduction and internal fixation at The First Affiliated Hospital of Xinjiang Medical University or The Johns Hopkins University School of Medicine. All subjects were screened using a detailed questionnaire, disease history, and physical examination.

### Mice

Mouse studies were conducted in the animal facility of The Johns Hopkins University School of Medicine, and procedures were performed under a protocol approved by the Institutional Animal Care and Use Committee of The Johns Hopkins University (Baltimore, Md., USA). STR/Ort mice were bought from Harlan Laboratories (Frederick, Md., USA), CBA/J mice and DMP1-Cre mice were bought from the Jackson Laboratory (Bar Harbor, Me., USA), TNF-α transgenic (hemizygous) mice were obtained from Taconic Biosciences (Hudson, N.Y., USA), and COX-2^{flox/flox} mice were provided by Harvey Herschman, PhD, at the University of California-Los Angeles (Los Angeles, Calif., USA).

To knock out COX-2 in the osteocytes of TNF-α transgenic mice, experiments were conducted that first crossed TNF-α transgenic mice with DMP1-Cre mice to create DMP-1Cre::TNF-α transgenic offspring. Then DMP-1Cre::TNF-α transgenic mice were crossed with COX-2^{flox/flox} mice to create TNF-α DMP1-Cre:: COX-2^{flox/flox} experimental mice and COX-2^{flox/flox} littermate controls.

CIA procedures were performed on 2-month-old mice using the methods described by Brand and colleagues (27). For the time-course experiments, CIA mice or the non-immunized controls were euthanized at 100 days after initial immunization.

### Cell Culture

Experiments isolated primary osteocytes from different mouse models using a protocol described previously (see, Watters, J. W., et al. Arthritis and rheumatism 56, 2999-3009 (2007)). Experiments collected subchondral bone from mice by carefully removing the attached soft tissue, then washing the bones with a-minimum essential medium (MEM)+ 10% penicillin and streptomycin to remove contaminants. Experiments were conducted that cut the bone into 1- to 2-mm lengths and used warmed collagenase solution (4 mg/ml type IA collagenase in α-MEM) to incubate bone pieces 3 times at 25° C. for 25 minutes. Then experiments were conducted that used warmed collagenase solution (4 mg/ml type IA collagenase in α-MEM) and ethylenediaminetetraacetic acid (EDTA) solution (5 mMEDTA solution in Dulbecco's phosphate-buffered saline [PBS] with 1% bovine serum albumin) to incubate bone pieces 2 times at 25° C. alternatively; experiments aspirated the solution and retained it for cell plating. The isolated primary osteocytes and minced bone pieces were cultured with primary bone cell culture medium (α-MEM with 5% fetal bovine serum, 5% calf serum, and 1% penicillin and streptomycin) in collagen-coated plates. Experiments were conducted that collected protein and the culture medium for Western blot and enzyme-linked immunosorbent assay (ELISA) analysis at 72 hours.

### ELISA and Western Blot

Experiments detected the concentration of serum and medium PGE2 level using a PGE2 ELISA kit (514010, Cayman Chemical, Ann Arbor, Mich., USA) according to the manufacturer's instructions. Total cell lysates were separated by SDS-PAGE (sodium dodecyl sulfate polyacrylamide gel electrophoresis) blotted on polyvinylidene fluoride membranes (MilliporeSigma, Temecula, Calif., USA). The membranes were blocked with 5% milk (170-6404, Bio-Rad Laboratories, Inc., Hercules, Calif., USA) and incubated with specific antibodies to COX-2 (ab15191, 1:20000, Abcam, Cambridge, Mass., USA), then reprobed with appropriate horseradish peroxidase-conjugated secondary antibodies. Blots were developed using a SuperSignal West Femto Maximum Sensitivity Substrate Kit (QJ222305, Thermo Fisher Scientific, Inc., Waltham, Md., USA) and exposed to x-ray films.

### μCT Analysis

Knee joints were dissected from mice, carefully removing the attached muscle, fixed for 4 hours with 4% paraformaldehyde at 4° C., and washed 3 times with ice-cold PBS. Experiments were then performed μCT analysis using high-resolution μCT (Skyscan 1172, Bruker microCT, Kontich, Belgium). The scanner was set at a voltage of 65 kV, a current of 154 μA, and a resolution of 5.8 μm per pixel. The image reconstruction software (NRecon, version 1.6, Bioz, Inc., Palo Alto, Calif., USA), data analysis software (CT Analyser, version 1.9, Bruker microCT) and 3-dimensional model visualization software (μCT Volume, version 2.0, Bruker microCT) were used to analyze the parameters of the distal femoral metaphyseal trabecular bone. Experiments were conducted that selected the trabecular bone of subchondral bone as the region of interest for analysis. Trabecular BV per TV and TBPf were measured.

### Histochemistry and Histomorphometry

Knee joints were dissected from mice, carefully removing the attached muscle and fixed overnight with 10% formalin at 4° C. After washing 3 times with ice-cold PBS, the samples were decalcified at 4° C. using 10% EDTA (pH 7.4) for 21 days and then embedded in paraffin. Four-micrometer-thick sagittal-oriented sections of the knee joint medial compartment were used for staining. The slides were processed for H&E, safranin O, and fast green staining. TRAP staining was performed using a standard protocol (Sigma-Aldrich, St. Louis, Mo., USA).

### Immunocytochemistry and Histomorphometry

Knee joints were dissected from mice, carefully removing the attached muscle and fixed overnight with 10% formalin at 4° C. After washing 3 times with ice-cold PBS, the samples were decalcified at 4° C. using 10% EDTA (pH 7.4) for 21 days and then embedded in paraffin. Four-micrometer-thick sagittal-oriented sections of the knee joint medial compartment were used for staining. The sections were stained with individual primary antibodies to COX-2 (ab15191, 1:100, Abcam), osterix (ab22552, 1:100, Abcam), OCN (M137, 1:100, Takara Bio Inc., Kusatsu, Shiga Prefecture, Japan), pSmad2/3 (sc-11769, 1:50, Santa Cruz Biotechnology Inc., Dallas, Tex., USA), MMP13 (ab3208, 1:50, Abcam) at 4° C. overnight. Experiments were conducted that used the horseradish peroxidase-streptavidin detection system (Dako) to detect immunoactivity. Then experiments were conducted that counterstained the sections with hematoxylin (Sigma-Aldrich). Experiments were conducted that counted the numbers of positively stained cells in 4 random visual fields in subchondral bone in 5 sequential sections per mouse in each group. For OCN staining experiments were conducted that normalized them to the number per millimeter of adjacent bone surface (N mm-1) in subchondral bone.

### Immunofluorescence Staining

Immunofluorescence staining was performed as described previously (see, Myers, L. K., et al. Arthritis & Rheumatism 43, 2687-2693 (2000)). Knee joints were dissected from mice, carefully removing the attached muscle, and fixed for 4 hours with 4% paraformaldehyde at 4° C. After washing 3 times with ice-cold PBS, the samples were decalcified at 4° C. using 0.5M EDTA (pH 7.4) for 24 h (2-month-old mice) and 48 h (4- and 6-month-old mice) with constant shaking. Then experiments were conducted that incubated the decalcified bones in 20% sucrose and 2% polyvinylpyrrolidone (PVP) solution for 24 h and embedded the tissues in 8% gelatin (porcine) in the presence of 20% sucrose and 2% PVP. Forty-micrometer-thick sagittal-oriented sections of the knee joint medial compartment were used for staining. The slides were stained with individual primary antibodies to CD31 (ab28364, 1:100, Abcam), endomucin (V.7C7, 1:50, Santa Cruz Biotechnology, Inc.) at 4° C. overnight. Then secondary antibodies conjugated with fluorescence were added, and slides were incubated at room temperature for 1 h while avoiding light. The sections were observed under a confocal microscope (LSM 780, Zeiss, Oberkochen, Germany, USA)

### Calcein Double-Labeling

To examine dynamic bone formation, mice were injected intraperitoneally with 0.08% calcein (Sigma-Aldrich, 20 mg/kg b.w.) 8 and 2 days before euthanasia. Calcein double-labeling in undecalcified bone slices was observed under a fluorescence microscope. Four randomly selected visual fields in the distal metaphysis of the femur were measured to test trabecular bone formation in subchondral bone.

### Von Frey Testing

Experiments were conducted that detected mechanical sensitivity by applying 0.008-g von Frey filaments (Stoelting Co., Wood Dale, Ill., USA) on the plantar surface of mouse hind paws. Experiments were conducted that used the number of paw withdrawals in 3 sets of 10 stimulations each to represent the mechanical allodynia level.

### Statistics

Data are presented as means±standard deviations. For comparisons between 2 groups, 2-tailed Student t tests were used. For comparisons among multiple groups (e.g., OARSI scores, bone mass, and microarchitecture among groups), 1-way ANOVA was used. All experiments were repeated at least 3 times, and representative experiments are shown. Differences were considered significant at P<0.05. All data analyses were performed using SPSS, version 15.0, software (IBM Corp., Armonk, N.Y., USA).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating, delaying progression of, or reducing the severity of one or more metabolic disorders characterized with increased COX-2 expression and/or PGE2 expression and/or EP4 expression in bone related cells, comprising administering to a subject in need thereof a therapeutically effective amount of an agent configured to inhibit and/or diminish COX-2 expression, and/or PGE2 expression, and/or EP4 expression in bone related cells.

2. The method of claim 1, wherein the administration results in one or more of the following in the bone cells: inhibited or reduced COX-2 expression; inhibited or reduced PGE2 expression; inhibited or reduced EP4 expression; inhibited or reduced aberrant subchondral bone remodeling and/or innervation; inhibited or reduced cartilage degeneration;
and inhibited or reduced joint destruction.

3. The method of claim 1, wherein the one or more metabolic disorders characterized with increased COX-2 expression and/or PGE2 expression and/or EP4 expression in bone related cells are selected from osteoarthritis (OA), rheumatoid arthritis (RA), cardiovascular disease, and diabetes.

4. The method of claim 1, wherein the agent configured to inhibit and/or diminish COX-2 expression, and/or PGE2 expression, and/or EP4 expression in bone related cells is a COX-2 inhibiting agent.

5. The method of claim 4, wherein the COX-2 inhibiting agent is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lornoxicam, nabumetone, and diclofenac, as well as pharmaceutically acceptable salts of each, pharmaceutically acceptable derivatives of each, prodrugs of each, or mixtures thereof.

6. The method of claim 1, wherein the therapeutically effective amount of an agent configured to inhibit and/or diminish COX-2 expression, and/or PGE2 expression, and/or EP4 expression in bone related cells comprises administering approximately 8 mg/kg for approximately 4 weeks.

7. The method of claim 1, wherein the bone related cells include one or more of osteoblast cells, osteocyte cells, and osteoclast cells.

8. The method of claim 1, wherein the subject is a mammalian subject.

9. The method of claim 1, wherein the subject is a human patient suffering from or at risk of suffering from a metabolic disorder characterized with increased COX-2 expression and/or PGE2 expression and/or EP4 expression in bone related cells.

10. The method of claim 1, wherein the agent configured to inhibit and/or diminish COX-2 expression, and/or PGE2 expression, and/or EP4 expression in bone related cells is co-administered with a drug known for treating metabolic disorders characterized with increased COX-2 expression and/or PGE2 expression and/or EP4 expression in bone related cells.

11. The method of claim 10, wherein the drug is one or more of a drug known for treating OA; a drug known for treating RA; a drug known for treating CVS; and a drug known for treating diabetes.

12. A kit comprising one or more agents configured to inhibit and/or diminish COX-2 expression, and/or PGE2 expression, and/or EP4 expression in bone related cells (e.g., agents sufficient to interfere with central nervous system related NK levels and function) and instructions for administering the agent to an animal. The kits may optionally contain one or more other therapeutic agents.

13. A method of inhibiting and/or reducing PGE2 expression in bone cells comprising exposing bone cells characterized with increased PGE2 expression (compared to an established normal expression level) a therapeutically effective amount of an agent configured to inhibit and/or diminish COX-2 expression, and/or PGE2 expression, and/or EP4 expression in bone related cells.

14. The method of claim 13, wherein the agent configured to inhibit and/or diminish COX-2 expression, and/or PGE2 expression, and/or EP4 expression in bone related cells is a COX-2 inhibiting agent.

15. The method of claim 14, wherein the COX-2 inhibiting agent is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lornoxicam, nabumetone, and diclofenac, as well as pharmaceutically acceptable salts of each, pharmaceutically acceptable derivatives of each, prodrugs of each, or mixtures thereof.

16. The method of claim 13, wherein the therapeutically effective amount of an agent configured to inhibit and/or diminish COX-2 expression, and/or PGE2 expression, and/or EP4 expression in bone related cells comprises administering approximately 8 mg/kg for approximately 4 weeks.

17. A method of inhibiting and/or reducing EP4 expression in bone cells comprising exposing bone cells characterized with increased EP4 expression (compared to an established normal expression level) a therapeutically effective amount of an agent configured to inhibit and/or diminish COX-2 expression, and/or PGE2 expression, and/or EP4 expression in bone related cells.

18. The method of claim 17, wherein the agent configured to inhibit and/or diminish COX-2 expression, and/or PGE2 expression, and/or EP4 expression in bone related cells is a COX-2 inhibiting agent.

19. The method of claim 18, wherein the COX-2 inhibiting agent is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lomoxicam, nabumetone, and diclofenac, as well as pharmaceutically acceptable salts of each, pharmaceutically acceptable derivatives of each, prodrugs of each, or mixtures thereof.

20. The method of claim 17, wherein the therapeutically effective amount of an agent configured to inhibit and/or diminish COX-2 expression, and/or PGE2 expression, and/or EP4 expression in bone related cells comprises administering approximately 8 mg/kg for approximately 4 weeks.

21. A method of inhibiting and/or reducing aberrant subchondral bone remodeling and/or innervation in bone cells comprising exposing bone cells characterized with aberrant subchondral bone remodeling and/or innervation a therapeutically effective amount of an agent configured to inhibit and/or diminish COX-2 expression, and/or PGE2 expression, and/or EP4 expression in bone related cells.

22. The method of claim 21, wherein the agent configured to inhibit and/or diminish COX-2 expression, and/or PGE2 expression, and/or EP4 expression in bone related cells is a COX-2 inhibiting agent.

23. The method of claim 22, wherein the COX-2 inhibiting agent is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lomoxicam, nabumetone, and diclofenac, as well as pharmaceutically acceptable salts of each, pharmaceutically acceptable derivatives of each, prodrugs of each, or mixtures thereof.

24. The method of claim 21, wherein the therapeutically effective amount of an agent configured to inhibit and/or diminish COX-2 expression, and/or PGE2 expression, and/or EP4 expression in bone related cells comprises administering approximately 8 mg/kg for approximately 4 weeks.

25. A method of inhibiting and/or reducing joint cell destruction in bone cells comprising exposing bone cells characterized with joint destruction a therapeutically effective amount of an agent configured to inhibit and/or diminish COX-2 expression, and/or PGE2 expression, and/or EP4 expression in bone related cells.

26. The method of claim 25, wherein the agent configured to inhibit and/or diminish COX-2 expression, and/or PGE2 expression, and/or EP4 expression in bone related cells is a COX-2 inhibiting agent.

27. The method of claim 26, wherein the COX-2 inhibiting agent is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, aspirin, acetaminophen, ibuprofen, flurbiprofen, ketoprofen, naproxen, oxaprozin, etodolac, indomethacin, ketorolac, lomoxicam, nabumetone, and diclofenac, as well as pharmaceutically acceptable salts of each, pharmaceutically acceptable derivatives of each, prodrugs of each, or mixtures thereof.

28. The method of claim 25, wherein the therapeutically effective amount of an agent configured to inhibit and/or diminish COX-2 expression, and/or PGE2 expression, and/or EP4 expression in bone related cells comprises administering approximately 8 mg/kg for approximately 4 weeks.
